(19) United States
(12) Patent Application Publication
(10) Pub. No.: US 2025/0152794 A1
(43) Pub. Date: May 15, 2025

(54) CONTROL OF A MACHINE FOR PERITONEAL DIALYSIS

(71) Applicant: GAMBRO LUNDIA AB, Lund (SE)

(72) Inventors: Per-Olof BORGQVIST, Lund (SE); Mattias HOLMER, Lund (SE); Karin BERGLING, Lund (SE)

(21) Appl. No.: 18/839,371

(22) PCT Filed: Mar. 7, 2023
(86) PCT No.: PCT/EP2023/055658

§ 371 Date: Aug. 16, 2024

(30) Foreign Application Priority Data
Mar. 9, 2022 (SE) 2250306-4

(51) Int. Cl. A61M 1/28; G16H 20/40; G16H 40/63

(52) U.S. Cl. CPC A61M 1/282; G16H 20/40; G16H 40/63; A61M 2202/0007; A61M 2202/0014; A61M 2202/04; A61M 2205/18; A61M 2205/3327; A61M 2205/3331; A61M 2205/3379; A61M 2205/502; A61M 2205/52

(57) ABSTRACT

A control device performs a method to generate control signals for a dialysis machine, which is configured to perform peritoneal dialysis, PD, treatment comprising one or more fluid exchange cycles in relation to a peritoneal cavity of a patient, to cause a transport of fluid and solutes through its peritoneal membrane. The method comprises: obtaining a target value of a treatment parameter for the PD treatment, obtaining a transport property of the peritoneal membrane, and configuring a transport model by use of the at least one transport property. The transport model defines the transport of fluid and solutes through the peritoneal membrane as a function of control parameters for the PD treatment. The method further comprises: evaluating the transport model to determine set values of the control parameters to achieve the target value, and generating the control signals in correspondence with the set values.

### TECHNICAL FIELD

The present disclosure relates generally to peritoneal dialysis, and in particular to techniques for configuring and operating a dialysis machine to perform a peritoneal dialysis treatment in relation to a patient.

### BACKGROUND ART

In the treatment of individuals suffering from acute or chronic renal insufficiency, dialysis therapy may be needed. One category of dialysis therapy is peritoneal dialysis (PD). In PD, a treatment fluid (“dialysis fluid”) is infused into the individual's peritoneal cavity via an implanted catheter, in a fill phase. The peritoneal cavity is lined by a peritoneal membrane (“peritoneum”) which is highly vascularized. The treatment fluid is allowed to reside in the peritoneal cavity in a dwell phase, whereupon fluid is extracted from the peritoneal cavity in a drain phase. Substances (solutes) are removed from the patient's blood mainly by diffusion across the peritoneum into the treatment fluid. Excess fluid (water) is also removed by osmosis through the peritoneum, by the treatment fluid containing osmotic agent. The removal of excess fluid is also known as ultrafiltration. A PD treatment may include any number of fluid exchange cycles, each comprising a fill phase, a dwell phase and a drain phase.

In so-called automated peritoneal dialysis (APD), the PD treatment is controlled by a dialysis machine, also known as a PD cycler. A healthcare professional determines set values for various control parameters of the PD cycler with the intention to achieve one or more treatment targets, for example in terms of ultrafiltration and/or removal of solutes such as urea. Examples of control parameters include composition of treatment fluid, treatment time, number and frequency of fluid exchange cycles, and fill volume per cycle. The determination of set values is made ad hoc and based on previous experience of the healthcare professional and possibly in view of general guidelines. There is currently no way of validating that the set values are reasonably correct in advance of a PD treatment or to guide the healthcare professional on suitable set values to be entered into the PD cycler in view of a treatment target.

The difficulty of determining proper set values for the PD cycler is aggravated by the fact that there is considerable variability between patients in solute transport capacity and ultrafiltration capacity of the peritoneum. Moreover, continuous exposure to treatment fluids may lead to functional alterations of the peritoneum over time. Therefore, it is standard procedure to perform peritoneal testing to assess the functionality of the peritoneum. There are numerous options for peritoneal testing, including the peritoneal equilibration test (PET). For example, a Standard PET test may be used to classify the patient into one of a plurality of transporter categories, such as High, High Average, Low Average, and Low. Based on this categorization, the caretaker may qualitatively determine set values for the PD cycler. PET tests may also be implemented to quantify a transport property of the peritoneum, such as its filtration capacity of water. Apart from resulting in a coarse and rather non-informative characterization of the peritoneum, the Standard PET test is complicated and requires significant expenditure of time and resources, for example to run tests, perform laboratory analysis, interpret data, etc. Standard PET is performed by medical staff and is time-consuming for the patient, who needs spend at least half a day at a dedicated clinic or a hospital.

Further, a technique of estimating the osmotic conductance to glucose (OCG) is presented in the article “Novel Method for Osmotic Conductance to Glucose in Peritoneal Dialysis”, by Giedre et al, published in Kid Int Rep 5 (11), pp 1974-1981, 2020. The OCG parameter may be seen to quantify the filtration capacity of water through the peritoneum.

### SUMMARY

It is an objective to at least partly overcome one or more limitations of the prior art.

One objective is to facilitate the task of configuring a dialysis machine to perform a PD treatment.

Another objective is to improve the performance of such a dialysis machine.

One or more of these objectives, as well as further objectives that may appear from the description below, are at least partly achieved by a control device for operating a dialysis machine, an arrangement for performing peritoneal dialysis, a computer-implemented method of generating control signals, and a computer-readable medium according to the independent claims, embodiments thereof being defined by the dependent claims.

A first aspect is a control device for operating a dialysis machine to perform a peritoneal dialysis treatment in relation to a patient, said dialysis machine being operable, based on control signals from the control device, to perform one or more fluid exchange cycles in relation to a peritoneal cavity of the patient to cause a transport of fluid and solutes through a peritoneal membrane in the peritoneal cavity. The control device comprises control circuitry configured to: obtain a target value of a treatment parameter for the peritoneal dialysis treatment; obtain at least one transport property of the peritoneal membrane of the patient; configure a transport model by use of said at least one transport property, the transport model defining the transport of fluid and solutes through the peritoneal membrane as a function of a plurality of control parameters for the peritoneal dialysis treatment performed by the dialysis machine; evaluate the transport model to determine set values of the plurality of control parameters to achieve the target value; and generate control signals for the dialysis machine in correspondence with the set values, said control signals causing the dialysis machine to perform the peritoneal dialysis treatment. The control circuitry is further configured to: obtain first data comprising a measured value of a concentration-related parameter of fluid in the peritoneal cavity at one or more time points during the peritoneal dialysis treatment; obtain second data indicative of fluid flow of treatment fluid into and/or out of the peritoneal cavity during the peritoneal dialysis treatment; and calculate, by use of the transport model and based on the first data and the second data, at least one updated transport property of the peritoneal membrane of the patient.

A second aspect is an arrangement for performing peritoneal dialysis. The arrangement comprises: a fluid circuit that is connectable to a peritoneal access of a patient for conveying treatment fluid to and from a peritoneal cavity; a dialysis machine configured to operate the fluid circuit; and a control device in accordance with the first aspect, or any embodiments thereof.

Third aspect is a computer-implemented method of generating control signals causing a dialysis machine to perform a peritoneal dialysis treatment in relation to a patient, said peritoneal dialysis treatment comprising one or more fluid exchange cycles in relation to a peritoneal cavity of the patient to cause a transport of fluid and solutes through a peritoneal membrane in the peritoneal cavity. The method comprises: obtaining a target value of a treatment parameter for the peritoneal dialysis treatment; obtaining at least one transport property of the peritoneal membrane of the patient; configuring a transport model by said at least one transport property, the transport model defining the transport of fluid and solutes through the peritoneal membrane as a function of a plurality of control parameters for the peritoneal dialysis treatment performed by the dialysis machine; evaluating the transport model to determine set values of the plurality of control parameters to achieve the target value; and generating the control signals for the dialysis machine in correspondence with the set values. The method further comprises: obtaining first data comprising a measured value of a concentration-related parameter of fluid in the peritoneal cavity at one or more time points during the peritoneal dialysis treatment; obtaining second data indicative of fluid flow of treatment fluid into and/or out of the peritoneal cavity during the peritoneal dialysis treatment; and calculating, by use of the transport model and based on the first data and the second data, at least one updated transport property of the peritoneal membrane of the patient.

A fourth aspect is a computer-readable medium comprising computer instructions which, when executed by one or more processors, cause the one or more processors to perform the method of the third aspect, or any embodiments thereof.

These aspects are based on the insight that a mathematical transport model for the peritoneal membrane may be used as a link between set values of a dialysis machine and one or more treatment parameters that represent the effect of the peritoneal dialysis (PD) treatment, as performed by a dialysis machine on a patient. This insight is applied in a technique that, in accordance with the foregoing aspects, enable the dialysis machine to be automatically controlled to perform a PD treatment to achieve a target value of at least one treatment parameter. The target value may be entered by a user of the dialysis machine (for example, a healthcare professional) in advance of a treatment session, to define a desired change of the physiological status of the patient when the treatment session is completed. For example, the target value may represent removal of fluid from the patient, or removal of one or more solutes from the patient. The technique defined by the foregoing aspects provides the technical advantage of improving the ability of the dialysis machine to achieve the target value through the PD treatment. A further advantage is that set values for the dialysis machine may be adapted to the patient, to account for the state of the patient's peritoneal membrane. This may be achieved by configuring the transport model with a patient-specific value of one or more transport properties of the peritoneal membrane. Such a patient-specific value may, for example, be determined as described in the Background section or be determined in accordance with the aspects for a preceding PD treatment of the patient.

Compared to conventional practice, the healthcare professional is at least partly relieved of the burden to determine appropriate set values for the dialysis machine. Further, the performance of the dialysis machine is improved in terms of its ability to achieve a desired end result of the peritoneal dialysis treatment. Also, the impact of human error when determining or entering set values for the dialysis machine is reduced or eliminated.

The foregoing aspects are also based on the insight that it is possible to validate the transport model based on the first and second validation data for the PD treatment, to verify that the transport model accurately represents the transport of fluid and solutes through the peritoneal membrane and, if deemed necessary, use the transport model to calculate an updated value of at least one transport property of the peritoneum. Thus, the aspects are inherently capable of providing an updated and patient-specific value of at least one transport property that is included in the transport model. This is a significant technical advance since it provides more detailed information about the status of the peritoneum than the conventional Standard PET test and without any major effort by medical staff. The aspects further open up the possibility of applying the updated transport property to improve the transport model. Such an improved transport model may be used for generating control signals for a future PD treatment performed by the dialysis machine, or by another dialysis machine, in accordance with the foregoing aspects. Alternatively or additionally, the improved transport model may be used for determining updated set values, and for generating updated control signals for the dialysis machine, during on-going PD treatment. This will improve the ability of the dialysis machine to reach the target value for the PD treatment.

As will be further described below, a “peritoneal dialysis treatment” or “PD treatment” may be a single PD session, a sequence of PD sessions, or part of a PD session, depending on implementation.

Still other objectives and aspects, as well as embodiments, features, technical effects and advantages may appear from the following detailed description, from the attached claims as well as from the drawings. It may be noted that any embodiment of the first aspect, as found herein, may be adapted and implemented as an embodiment of the second to fourth aspects.

### BRIEF DESCRIPTION OF THE DRAWINGS

FIG. 1 illustrates an example arrangement for automated peritoneal dialysis (APD).

FIGS. 2A-2B are example plots of intraperitoneal volume versus time in APD therapy, and FIG. 2C is an example diagram of input data and output data for a control device in an arrangement for APD.

FIG. 3 illustrates transport processes affecting solute concentration and fluid volume in a peritoneal cavity.

FIG. 4A is a flowchart of an example method of generating control signals for a dialysis machine, and FIGS. 4B-4E are flowcharts of example procedures in the method of FIG. 4A.

FIG. 5 is a combination of plots of intraperitoneal conductivity, glucose concentration and fluid volume versus time calculated by use of a transport model.

FIGS. 6A-6C are flowcharts of monitoring methods performed during PD treatment, and FIG. 6D is a graph of example ranges for triggering a recalculation of a transport property of the peritoneal membrane.

FIGS. 7A-7B are block diagrams of example computation arrangements for generating control signals, FIG. 7C is a block diagram of an example implementation of a peritoneum transport model in a computation arrangement, and FIGS. 7D-7E are block diagrams of circuitry for calculating a current value of a treatment parameter in the implementation of FIG. 7C.

FIGS. 8A-8B are flowcharts of an example method of determining a reference value of the fluid volume in the peritoneal cavity for use in the method of FIGS. 4A-4C.

FIG. 9 is an example plot of intraperitoneal volume and data samples taken over time during a test cycle.

FIG. 10 is a block diagram of an example computation arrangement for generating the reference value in accordance with the method of FIGS. 8A-8B.

FIGS. 11-13 depict results of calculations in accordance with embodiments.

FIG. 14 is a block diagram of an example machine that may implement the methods, procedures and functions described herein.

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more”, even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

As used herein, the terms “multiple”, “plural” and “plurality” are intended to imply provision of two or more elements. The term “and/or” includes any and all combinations of one or more of the associated listed elements.

It will furthermore be understood that although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure.

Well-known functions or constructions may not be described in detail for brevity and/or clarity. Unless otherwise defined, all terms (including technical and scientific terms) used herein have the same meaning as commonly understood by one of ordinary skill in the art to which this disclosure belongs.

As used herein, “PD session”, “treatment session” and “session” refer to a machine-controlled PD treatment which starts by the patient being connected to a dialysis machine and ends by the patient being disconnected from the dialysis machine. A PD session is thus a temporally isolated event.

Like reference signs refer to like elements throughout.

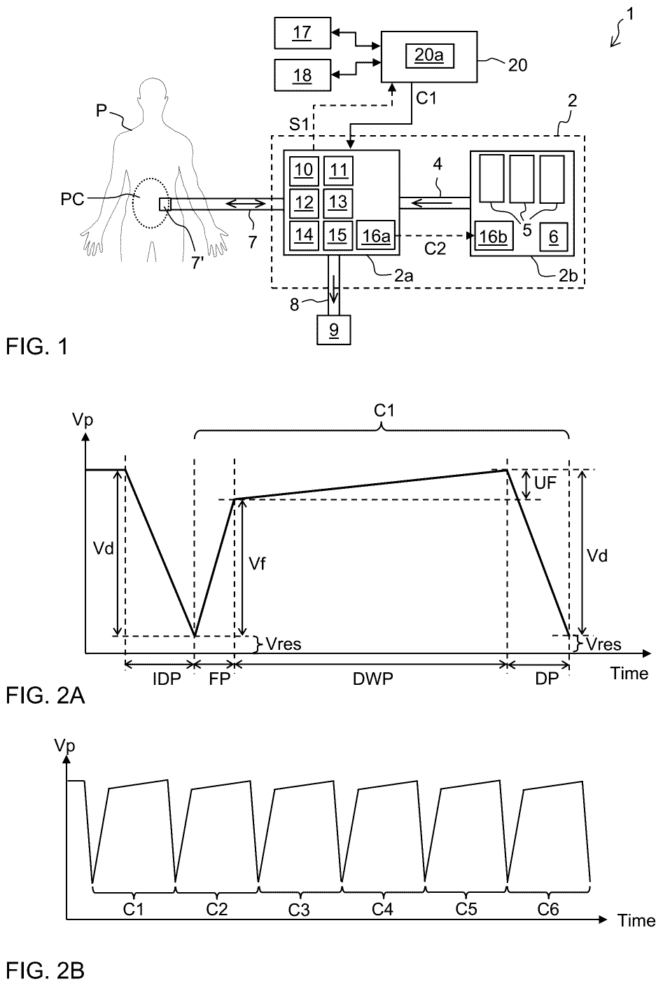

FIG. 1 is a schematic view of an example arrangement 1 for peritoneal dialysis treatment. The arrangement (or system) 1 is generally intended for on-site treatment of a patient P with proper treatment fluid(s). Specifically, the PD arrangement (PDA) 1 is designed for treating patients suffering of renal insufficiency, by automated peritoneal dialysis (APD).

In the illustrated example, the PDA 1 comprises a dialysis management unit (DMU) 2a and a fluid supply unit (FSU) 2b. The FSU 2b is connected to the DMU 2a by a supply line 4 and is operable to supply fresh treatment fluid to the DMU 2a. In the example of FIG. 1, the FSU 2b comprises three containers 5, which are arranged for fluid connection to the supply line 4, and a fluid control arrangement 6, which is operable to direct and pump a treatment fluid of a specific composition into the supply line 4. The fluid control arrangement 6 may comprise a combination of a fluid circuit, one or more fluid pumps, and one or more valves. The number of containers 5 in the FSU 2b may differ depending on the treatment fluids to be infused into the peritoneal cavity (PC) of the patient P during the course of the PD treatment. For example, different containers may hold treatment fluid of different composition. In an alternative, the FSU 2b is configured for on-line preparation of treatment fluid, batch-wise or on-demand, for example by mixing purified water and one or more concentrates.

A patient line 7 extends from the DMU 2a to a catheter or other access device 7′, which is implanted in the patient P. A drain line 8 extends from the DMU 2a to a drain 9 or, alternatively, a receptacle for spent treatment fluid. As used herein, spent treatment fluid refers to any treatment fluid that has resided inside the PC. The spent treatment fluid is also known as “effluent” in the art.

The DMU 2a is operable to perform a PD treatment comprising one or more fluid exchange cycles. The respective exchange cycle comprises a sequence of a fill phase, a dwell phase and a drain phase. In the fill phase, the DMU 2a is operated to pump fresh treatment fluid through the patient line 7 into the PC of the patient P. In the dwell phase, the treatment fluid resides in the PC. In the drain phase, the DMU 2a is operated to pump spent treatment fluid from the PC, via the patient line 7 and the drain line 8 to the drain 9. Although not shown, it is conceivable to have separate patient lines 7 for in-going and out-going treatment fluid.

The treatment fluid may comprise at least one osmotic agent. As is well-known in the art, the osmotic concentration of the treatment fluid relative to the blood determines to what extent fluids are exchanged between the treatment fluid and the blood. A high osmotic concentration in the treatment fluid creates a high gradient. In any of the embodiments described herein, the osmotic agent may be, or include, glucose (or polyglucose), L-carnitine, glycerol, icodextrin, or any other suitable agents. Alternative osmotic agents may be fructose, sorbitol, mannitol and xylitol. It is noted that glucose is also sometimes named as dextrose in the PD field. The term glucose is herewith intended to comprise dextrose.

The structure and functionality of the DMU 2a is implementation-specific. For this reason, FIG. 1 only schematically depicts some ordinary structural components of a DMU 2a. In the illustrated example, the DMU 2a comprises a fluid control arrangement 10, which is operable to direct and pump fresh treatment fluid into the patient line 7, withdraw spent treatment fluid from the patient line 7, and pump the spent treatment fluid into the drain line 8. The fluid control arrangement 10 may comprise a combination of a fluid circuit, one or more fluid pumps, and one or more valves. The DMU 2a further comprises a heater 11, which is operable to adjust the temperature of the fresh treatment fluid to a predefined temperature before it is pumped into the PC. The temperature is measured by a temperature sensor 12. A flow meter 13 is arranged to measure the flow rate of fluid pumped into and out of the patient line 7, and thus to and from the PC. In an alternative, if the treatment fluid is pumped to and from the PC by one or more volumetric pumps with known stroke volume, the flow rate may be measured by counting the number of pump strokes performed by the respective volumetric pump (“volumetric monitoring”) and the flow meter 13 may be omitted. Thus, any reference the flow meter 13 in the following may be replaced or supplemented by volumetric monitoring. A pressure sensor 14 is arranged to measure hydraulic pressure in the patient line 7 or in the fluid control arrangement 10. The measured hydraulic pressure may be used to control the fill phase and/or the drain phase. It may also be used for detection of operational errors during a PD session. At least one sensor 15 is arranged to measure a property of treatment fluid. The measured property is a concentration-related parameter, CRP. In some embodiments, the CRP sensor 15 is a conductivity sensor for measuring the electrical conductivity of a passing fluid. In some embodiments, the CRP sensor 15 is a concentration sensor for measuring a concentration of one or more substances in a passing fluid. The DMU 2a may be configured to selectively direct either fresh treatment fluid or withdrawn (spent) treatment fluid through one and the same CRP sensor 15. Alternatively, the DMU 2a may comprise a first CRP sensor arranged to sense a property of withdrawn treatment fluid, and a second CRP sensor arranged to sense a property of fresh treatment fluid.

In the illustrated example, the DMU 2a and the FSU 2b comprise a respective local controller 16a, 16b. The local controllers 16a, 16b are configured to operate the components the DMU 2a and the FSU 2b, respectively, in accordance with a respective operational scheme. As indicated by a dashed arrow, the local controller 16a may also generate control signals C2 for the local controller 16b to synchronize the FSU 2b with the DMU 2a and/or to cause the FSU 2b to provide a treatment fluid with a specific composition.

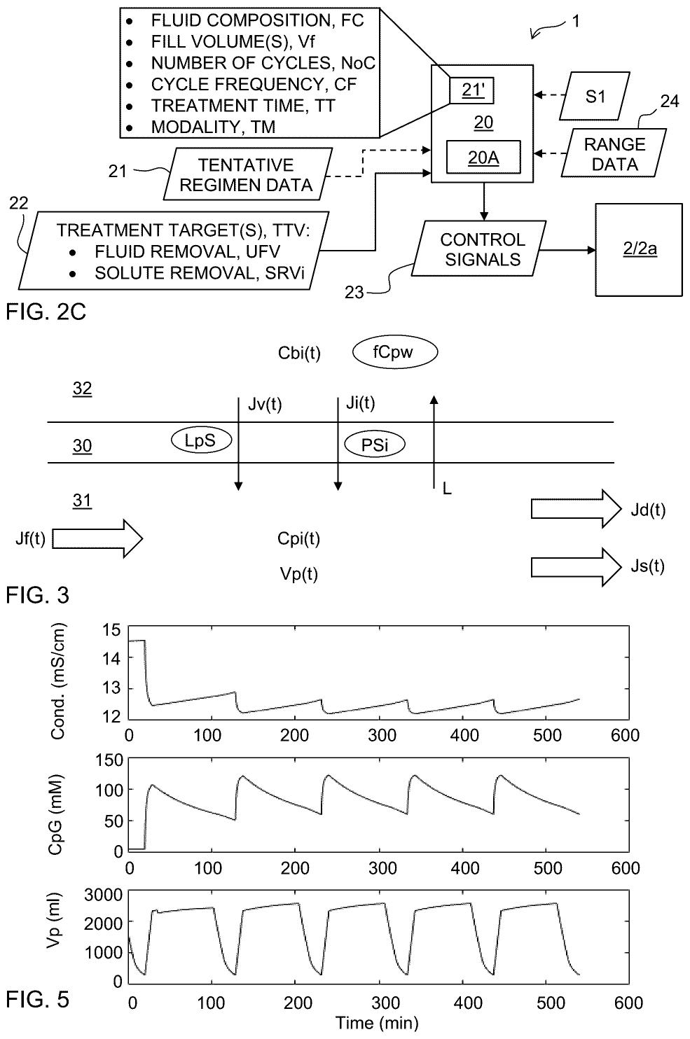

In FIG. 1, the PDA 1 further comprises a control device 20, which comprises control circuitry 20A for controlling the operation of the control device 20. The control circuitry 20A may comprise one or more processors and computer memory (cf. FIG. 14). The control device 20 is configured to determine a so-called regimen for the PD treatment. The regimen is determined in advance of a PD session, but may also be updated during an on-going session. The regimen defines the operational scheme to be performed by the DMU 2a during a PD session. The regimen will be described further below with reference to FIG. 2C. In FIG. 1, the control device 20 is configured to generate and provide control signals C1, which represent the regimen, to the DMU 2a. The local controller 16a is thereby caused to configure its operational scheme in accordance with the regimen. As indicated by a dashed arrow, the control device 20 may be configured to receive sensor data S1 from the DMU 2a. The sensor data S1 may include CRP values measured by the CRP sensor 15 and/or fluid flow data measured by the flow meter 13 or obtained by volumetric monitoring. The control device 20 may be configured to determine the regimen based on the sensor data S1.

In the illustrated example, the control device 20 is electrically connected to a user interface (UI) 17. The term “user interface” is intended to include any and all devices that are capable of performing guided human-machine interaction comprising presentation of information and receipt of input. Thus, the UI 17 may comprise a combination of a presentation device and data entry hardware. The presentation device may comprise one or more of a display, speaker, projector, lamps, etc. The data entry hardware may include one or more of a keyboard, keypad, computer mouse, control buttons, touch panel, microphone and voice control functionality, camera and gesture control functionality, etc. In one implementation, the UI 17 is or comprises a touch-sensitive display, also known as touch screen.

Further, the control device 20 may be electrically connected to an external memory 18, which may be a local or remote memory device for data storage. In some embodiments, the external memory 18 is part of a cloud-based service.

It should be noted that the DMU 2a may be composed of a re-usable machine part and a disposable part, as is well known in the art. The machine part of the DMU 2a is referred to as a dialysis machine or a “cycler” herein. The disposable part may define the fluid circuit of the fluid control arrangement 10 and is connected to or includes the patient line 7 and the drain line 8. The disposable part is mounted in engagement with the machine part before PD treatment, to “configure” the DMU 2a, and is discarded when the PD session is completed. The FSU 2b may also be composed of a re-usable machine part and a disposable part. For example, one or more of the containers 5 may be replaceable.

In some embodiments, the FSU 2b is integrated with the DMU 2a, forming an enhanced DMU 2. The resulting machine part is a dialysis machine or cycler with integrated fluid generation capability. Such a machine part may also be combined with a disposable part as described above.

In some embodiments, the control device 20 is integrated as a part of the DMU 2a or the enhanced DMU 2.

It is also conceivable that the local controller 16a is omitted and that the control device 20 implements the functionality of the local controller 16a. Likewise, the control device may implement the functionality of the local controller 16b.

The CRP sensor 15 may be included in the machine part of the DMU 2a or in the disposable part. If included in the machine part, the DMU 2a may comprise a cleaning unit (not shown) which is operable to perform a rinsing and/or disinfection of the CRP sensor 15. Alternatively, the CRP sensor 15 may alternatively be arranged in the patient line 7 or the drain line 8.

FIG. 2A schematically represents part of a PD session in terms of intraperitoneal volume, IPV, as a function of time. IPV designates the amount of fluid that resides in the PC. IPV is also referred to as Vp or “fluid volume” herein. Despite its connotation, IPV may be defined in terms of either volume or mass.

In a PD session, the PDA 1 is fluidly connected to the patient P, by the patient line 7 being connected to the access device 7′ (FIG. 1), whereupon the PDA 1 is operated to perform one or more fluid exchange cycles (“cycles”), optionally after an initial drain phase. For example, the initial drain phase may be performed if the patient carries a large fluid volume in the PC at start of the session. This happens when a previous session is terminated with a fill phase, i.e., an infusion of treatment fluid into the PC. After completion of the cycles, the patent P may be fluidly disconnected from the PDA 1.

The part of a PD session in FIG. 2A comprises an initial drain phase (IDP), which is followed by a fluid exchange cycle (C1). The cycle C1 consists of a fill phase (FP), a dwell phase (DWP) and a drain phase (DP), performed in sequence. In IDP, a fluid volume Vd is withdrawn (“extracted”) from the PC, by operation of the fluid control arrangement 10, and conveyed to the drain 9 (FIG. 1). At completion of IDP, a residual amount Vres of spent treatment fluid is left in the PC. In FP, a fluid volume Vf of fresh treatment fluid is infused into the PC, by the fluid control arrangement 10 conveying fresh treatment fluid, generated by the FSU 2b into the patient line 7 (FIG. 1). In DWP, the treatment fluid is left to reside in the PC. In DP, a fluid volume Vd is extracted from the PC, leaving a residual volume, Vres. This residual volume may, and typically will, differ from the residual volume after IDP. Depending on the osmotic force gradient, transport of fluid from the patient's blood into the PC or vice versa via the peritoneal membrane, known as ultrafiltration (UF), can be either positive, increasing IPV, or negative, decreasing IPV. During FP, DWP and DP, UF is generally positive, increasing PC volume due to the osmotic agent in the treatment fluid. For illustration purposes, the effect of UF is only indicated for DWP in FIG. 2A.

The extraction of fluid volume Vd may be controlled in different ways. In one implementation (“fixed Vd extraction”), the fluid volume Vd is deterministic and given by a set value, and the fluid control arrangement 10 is operated to (if possible) extract Vd, as measured by the flow meter 13. Fixed Vd extraction is for example used in tidal PD or in an IRPC (below). In another implementation (“maximized Vd extraction”), the fluid control arrangement 10 is operated to extract as much fluid as possible from the PC. For example, the IDP/DP may be terminated when the fluid control arrangement 10 is unable to extract further fluid from the PC, for instance indicated by a low flow rate.

FIG. 2B illustrates IPV as a function of time during a PD session consisting of an initial drain phase followed by six consecutive fluid exchange cycles C1-C6. At the onset of each cycle, there is a residual volume in the PC (cf. Vres in FIG. 2A). This residual volume may be unknown and may differ from cycle to cycle.

FIG. 2C is a schematic overview of the PDA 1 in FIG. 1. The PDA 1 comprises a DMU 2 or 2a, which receives control signals 23 from the control device 20. The control signals 23 correspond to C1 in FIG. 1 and cause the DMU 2/2a to perform a PD session, for example as shown in FIG. 2A or FIG. 2B. As noted in the Background section, a conventional PDA 1 is operated based on set values determined by a healthcare professional, for example a physician. Conventionally, the set values are entered into the PDA 1 as a “regimen” that defines how an upcoming session is to be performed, and the PDA 1 is then operated in adherence to the regimen. The regimen may be seen as defining set values of control parameters for the PDA 1. The present disclosure, on the other hand, provides a technique of allowing the physician to specify a target value of one or more treatment parameters to be achieved by an upcoming session, whereupon the control device 20 automatically computes and applies a regimen that meets the target value, possibly subject to confirmation by the physician. As used herein, a target value of a treatment parameter (“treatment target”) defines a desired change of the patient's physiological status. Conventionally, the effectiveness of a PD session is reliant on ability of the physician to determine a proper regimen, which is typically done based on experience and rules of thumb. By contrast, the proposed technique provides a significant improvement in that it actively controls the PD session to achieve a desired change of the patient's physiological status. It is realized that the proposed technique has the potential of improving PD treatment, reducing the workload of physicians, reducing the risk for human errors, etc. In some embodiments, the technique allows the physician to enter a regimen, in accordance with conventional practice, whereupon the control device 20 evaluates the regimen and adjusts one or more control parameters to meet the treatment target. In some embodiments, the technique allows the physician to designate some control parameters as fixed and others as adjustable. When the physician has entered set values for the fixed control parameters, the control device 20 determines the set values for the adjustable control parameters, in view of the entered set values, to meet the treatment target.

The control device 20 is configured to determine a regimen for an upcoming PD session and provide corresponding control signals 23 to the DMU 2/2a. The regimen is determined in accordance with a regimen definition 21′, which is available to the control device 20. As used herein, a “regimen definition” specifies control parameters of the DMU 2/2a, and a “regimen” specifies set values of the control parameters to be applied by the DMU 2/2a in an upcoming PD session.

FIG. 2C shows an example of a regimen definition 21′ comprising a plurality of control parameters: FC, Vf, NoC, CF, TT and TM. Control parameter FC defines the composition of the fresh treatment fluid to be infused during the respective fill phase. The composition may be fixed or time varying. Control parameter Vf defines the fill volume (cf. FIG. 2A) and may or may not differ between cycles. Control parameter NoC defines the number of cycles in the session. Control parameter CF defines the cycle frequency in the session, which corresponds to the duration of the respective cycle. Control parameter TT defines the total treatment time, which corresponds to the duration of the session. Control parameter TM defines the modality of PD to be used in the session. In one example, the control parameter TM defines that the session should be completed by a drain phase (cf. FIGS. 2A-2B) or a fill phase. In another example, the control parameter TM defines the drain volume Vd for each drain phase. For example, Vd may set to result in a partial drain of the PC, as used in tidal PD. In another example, the control parameter TM may define that the session should involve continuous flow PD (CFPD) or any other PD modality. FIG. 2C is merely given as an example and the regimen definition 21′ may include fewer or additional control parameters. For example, some of the control parameters may be given by default values or be inherent to the PDA 1 and need not be included in the regimen definition 21′. Further, if the session consists of a single cycle (FIG. 2B), control parameter CF may be omitted.

As indicated in FIG. 2C, the control device 20 may receive tentative regimen data 21, which defines at least part of a regimen for the upcoming PD session and may be entered by a physician via the UI 17 (FIG. 1) or retrieved from memory. The tentative regimen data 21 comprises one or more values of control parameters in the regimen definition 21′. The regimen data 21 may also indicate a selection of control parameters that are allowed be modified by the control device 20.

The control device 20 is configured to obtain one or more treatment targets, TTV. The treatment target may be entered by the physician into the control device 20, for example through the UI 17 (FIG. 1). In one example, the treatment target represents net water transport through the peritoneum during the session and is designated by UFV. The treatment target UFV thereby designates the total amount of fluid extracted from the patient during the session. The treatment target UFV effectively corresponds to a decrease in weight of the patient during the session, assuming that the patient does not urinate or ingest fluid during the session. In another example, the treatment target represents the accumulated amount of one or more solutes removed from the patient during the session and is designated by SRVi. The treatment target SRVi may be given for any solute. In a non-limiting example, the solute is urea or creatinine, which are commonly used to quantify the effectiveness of dialysis. For example, the treatment target SRVi may be given in any format that is conventionally used to quantify solute removal, including but not limited to clearance, Kt/V, urea reduction ratio (URR), etc. For example, Kt/V for the PD session may be given by SRVi for urea (or creatinine) divided by the product of the total body water in the patient and the plasma concentration of urea (or creatinine) in the patient, as is well-known to the person of ordinary skill in the art. A weekly Kt/V may be derived by multiplying Kt/V by the number of PD sessions performed per week.

It is realized that both UFV and SRVi correspond to a change in the physiological status of the patient. Depending on implementation, the control device 20 may be configured to operate on UFV or SRVi, or both, to determine the control signals 23.

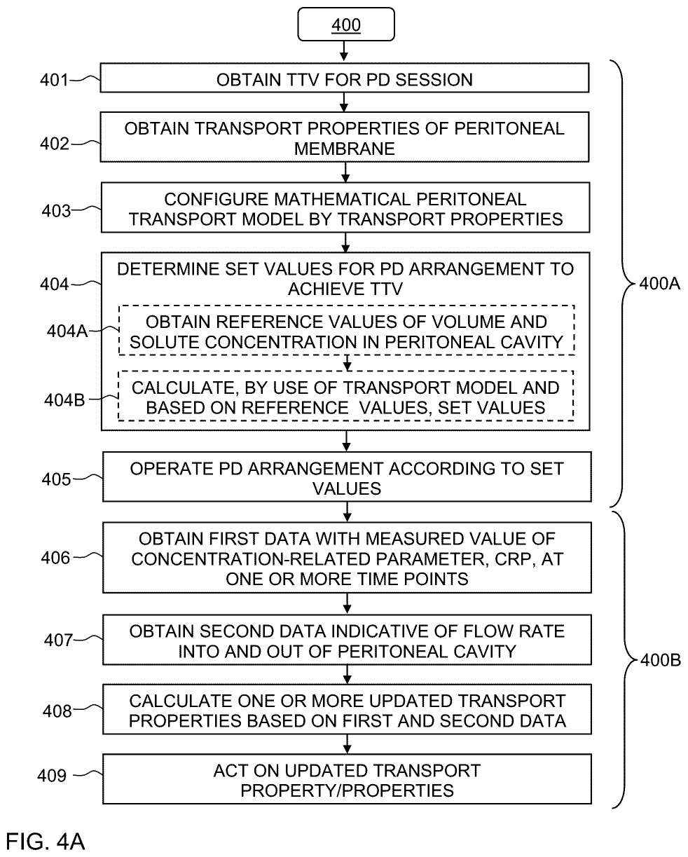

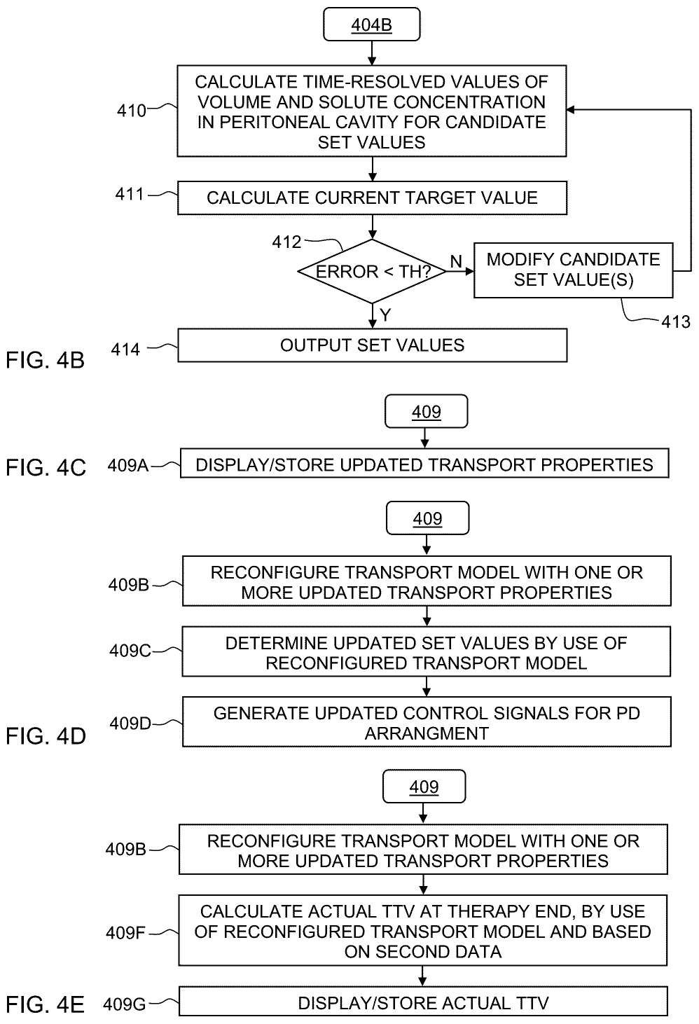

As shown in FIG. 2C, the control device 20 may also obtain range data 24, which defines allowable ranges for one or more of the control parameters in the regimen definition 21′. The use of the range data 24 will be described below with reference to FIG. 4B.

FIG. 3 is a schematic view of a peritoneal membrane (“peritoneum”) 30 that separates the PC 31 from the blood side 32 of the patient. In FIG. 3, the concentration of a respective solute (i) on the blood side 32 as a function of time is denoted Cbi(t), IPV as a function of time is denoted Vp(t), and the concentration of the respective solute (i) in the treatment fluid as a function of time is denoted Cpi(t). The solutes may include, without limitation, sodium, potassium, calcium, magnesium, lactate, phosphate, albumin, bicarbonate, urea, creatinine, chloride, etc. The solutes may also include an osmotic agent, for example according to any of the examples hereinabove. In the following examples, the osmotic agent is assumed to be glucose. FIG. 3 also depicts processes that may affect Vp(t) and Cpi(t). The processes comprise fluid flows through the access 7′, including a flow of treatment fluid into the PC 31 via the access 7′ during the fill phase(s), denoted Jf(t), a flow of treatment fluid out of the PC 31 via the access 7′ during the drain phase(s), denoted Jd(t), and a loss of treatment fluid from the PC 31 if and when fluid samples (see below) are taken of the treatment fluid in the PC 31 via the access 7′, denoted Js(t). The processes further comprise fluid flows through the peritoneum 30, including a total water flow, denoted Jv(t), a solute flow of the respective solute, denoted Ji(t), and a lymphatic flow, denoted L. It should be understood that the respective substance may move in either direction across the membrane 30 and that the arrows in FIG. 3 merely indicate a direction corresponding to a positive sign for the respective flow. In the following, it is assumed that the flow variables Jf, Jd and Js are given in ml/min, and the flow variable Ji is given in mmol/min.

The peritoneum 30 may be characterized by its transport properties. FIG. 3 indicate two such transport properties, LpS and PS. The property LpS is a hydraulic conductance of the membrane 30 and is also known as fluid permeability or ultrafiltration coefficient. The property LpS may, for example, be expressed in mL/min/mmHg. The property PS is a permeability-surface area product, which is also known as mass transfer area coefficient or diffusive mass transfer area coefficient and which represents the flow of a solute (molecule) through the membrane 30. The property PS may, for example, be expressed in mL/min. The property PS generally differs between solutes and is therefore denoted PSi herein. In some embodiments, it is assumed that there is a predefined relation between the PSi for all solutes, so that the PSi for any solute may be computed if the PSi for one solute is known. This assumption will facilitate the calculations, since determining PSi for a plurality of different solutes may be reduced to determining a scaling factor, fPS, which scales a generic set of PS values for the different solutes, PSiG, according to: PSi=fPS. PSiG, where PSIG is the generic PS value for solute i. The generic set represents the above-mentioned predefined relation and may be seen to include PS values representative of a “generic patient”. When fPS has been determined, any PSi may be determined by scaling the corresponding PSiG with fPS.

FIG. 3 indicates a further scaling factor, fCpw, which is representative of the tonicity of the patient and may represent the patient's deviation from an isotonic status. In some embodiments, to avoid the need for taking serum samples of the patient, the blood concentrations Cbi(t) of the solutes may be set to fixed (time-invariant) values for a standard (nominal) patient. To account for the fact that the tonicity may differ between patients, fCpw may be applied to scale the nominal blood concentration values. Thus, the scaling factor fCpw may be seen to account for the patient's actual plasma water fraction.

In some embodiments, a regimen for a PD session is determined by use of a mathematical model of the composition of the fluid in the PC 31. The mathematical model is a kinetic model that depends on the transport properties of the peritoneum 30 and accounts for the time dynamics of the flows of treatment fluid represented by Jf(t), Jd(t) and Js(t) in FIG. 3, as well as the time dynamics of the fluid flow Jv(t) and the solute flows Ji(t) through the membrane 30. These embodiments rely on the insight that the composition of the fluid in the PC is affected by the regimen and that the physiological status of the patient is in turn affected by the composition of the fluid in the PC 31 during the course of the session. Based on this insight, the kinetic model is configured as a transport model that defines the transport of fluid and solutes through the peritoneum as a function of control parameters in the regimen for a PD session. Such a transport model may be processed for determining a regimen that results in a desired change of the patient's physiological status.

FIG. 4A is a flowchart of an example method 400 of generating control signals causing a PDA to perform a PD treatment in relation to a patient. The method 400 may be executed by the control device 20 in FIGS. 1 and 2C. The control method 400 comprises a setup procedure 400A, which is performed to determine set values for the PDA. The set values are applied to generate control signals for operating the PDA to perform PD treatment. The control method 400 also comprises a validation procedure 400B, which is performed during PD treatment to validate the transport properties used by the setup procedure 400A and optionally take appropriate action. The setup procedure 400A will now be described in detail. The validation procedure 400B is described further below.

The example setup procedure 400A in FIG. 4A comprises steps 401-405. In step 401, one or more treatment targets TTV are obtained (cf. 22 in FIG. 2C). In step 402, transport properties of the peritoneal membrane are obtained. The transport properties may include LpS and PSi as described above. The transport properties may be entered by the user via the UI 17 or be retrieved by the control device 20 from internal memory or from an external memory (cf. 18 in FIG. 1). At least one of the transport properties is specific to the patient. The respective patient-specific transport property may be determined by any known technique based on measurements on the patient, for example as described in the Background section. A further technique of estimating patient-specific transport properties by use of the kinetic model is described further below. In step 403, the transport model is configured by the transport properties obtained in step 402. The model is “configured” by defining values of variables in the model, here variables that represent the transport properties. It is to be understood that step 403 may configure the transport model by further values, for example the scaling factor, fCpw, which may also be patient-specific and obtained in step 402. In step 404, the transport model is evaluated to determine set values (“control parameter values”) of a plurality of control parameters to achieve the treatment target(s) TTV obtained in step 401. The model is “evaluated” by adjusting the set values in accordance with an optimization procedure until the model achieves the respective treatment target. Any conventional optimization procedure may be used in step 404. In step 405, the PDA 1 is operated in accordance with the set values. In the context of FIGS. 1 and 2C, step 405 comprises generating the control signals 23 for the DMU 2/2a in correspondence with the set values. Step 405 may further comprise presenting the set values to the user, via the UI 17. The user may also be given the option to accept or reject the presented set values.

In some embodiments, step 401 involves the user entering UFV and/or SRVi via the UI 17 (FIG. 1). Alternatively, UFV may be calculated based on weight data for the patient, which is entered by the user via the UI 17 or otherwise obtained by the control device 20. The weight data may comprise an initial weight of the patient before the session, and a target weight of the patient at end of the session, and UFV may be calculated as a function of the difference between the current weight and the target weight. It is conceivable that SRVi is calculated in corresponding manner, if initial and target concentrations of the related solute are known or entered.

As indicated by dashed lines in FIG. 4A, step 404 may comprise a step 404A of obtaining reference values of fluid volume and solute concentration in the PC, and a step 404B of calculating the set values by use of the transport model and based on the reference values. The reference values are given at a respective reference time point. In step 404B, the reference values form boundary conditions that are used to “anchor” the transport model to values of fluid volume and solute concentration in the PC at the reference time point.

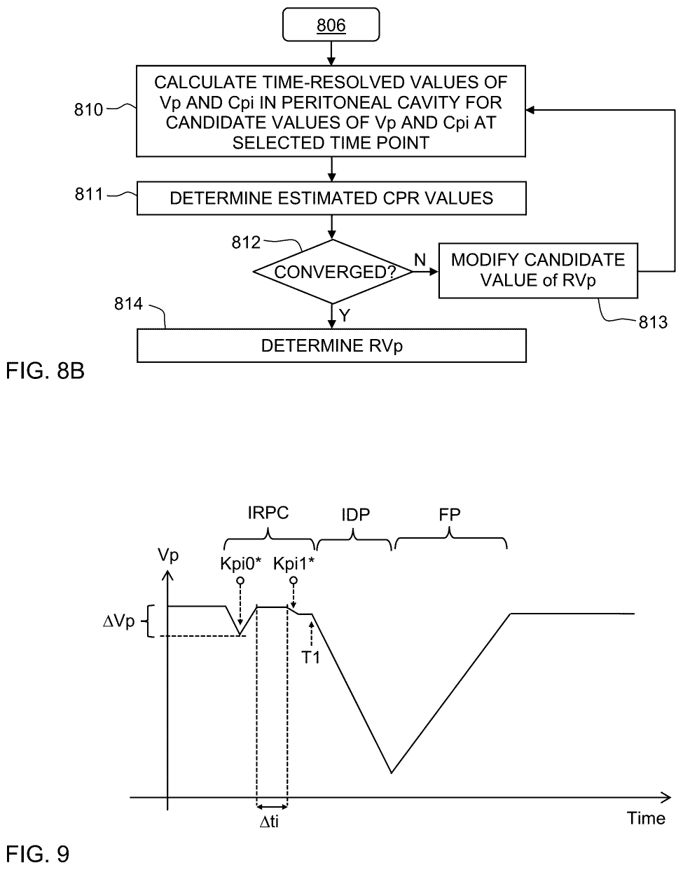

The reference values may be measured or estimated, or may be predefined values. The predefined values may be generic or patient-specific. In the example of solute concentration, a reference value may be obtained by extracting a sample of fluid from the PC 1 at one or more selected time points, measuring a CRP value of the respective sample by use of the CRP sensor 15 (FIG. 1), and estimating the solute concentration at the reference time point based on the CRP value(s). In the example of fluid volume, the residual volume Vres may be determined and used a reference value. For example, the residual volume after the initial drain phase may be estimated by use of a dilution formula and based on Vf and measured conductivity before and after a fill phase, for example as described in EP2623139. An alternative technique of estimating a reference value of fluid volume is described below with reference to FIGS. 8A-8B.

FIG. 4B is a flowchart of an example procedure that may be part of step 404B. In step 410, time-resolved values of fluid volume and solute concentration in the PC are calculated, by use of the transport model, for candidate set values of the control parameters. It is recalled that the fluid volume corresponds to Vp(t) in FIG. 3 and that the solute concentration corresponds to Cpi(t) in FIG. 3. The time-resolved values represent fluid volume and solute concentration at discrete time points during the PD session to be performed, given the candidate set values. The candidate set values that are used in step 410 define a tentative regimen and may be at least partly entered by the user and/or given by default values stored in a memory. The time-resolved values are calculated by operating the transport model, as configured in step 403, on the candidate set values while accounting for the boundary conditions defined by the reference values, as obtained in step 404A. In step 411, the time-resolved values are processed for calculation of a resulting (“current”) target value of the treatment parameter(s). In step 412, the difference (“error”) between the current target value and the corresponding treatment target TTV, as obtained in step 401, is evaluated in relation to an error threshold, TH. If the difference exceeds TH, one or more of the candidate set values is modified (updated) in step 413, in accordance with the optimization procedure. If a plurality of current target values (for different target parameters) are calculated in step 411, step 412 may calculate a corresponding plurality of differences and compare an aggregation of the calculated differences to the error threshold, TH. The resulting candidate set values are then again processed in step 410. Steps 410-413 are repeated until the difference no longer exceeds TH. At this time, the procedure proceeds to step 414, in which the last candidate set values generated by step 413 are output as set values for use by step 405.

As noted hereinabove, some control parameters may be designated as modifiable while others are designated as fixed. For example, the method 400 may comprise a step (not shown) that allows the user to select, via the UI 17, the control parameters to be fixed and modifiable, respectively. Alternatively or additionally, one or more control parameters may be predefined to the control device 20 as fixed or modifiable. In some embodiments, only candidate set values of modifiable control parameters are modified in step 413, while candidate set values for fixed control parameters remain unchanged. Further, in some embodiments, the method 400 comprises a step (not shown) that allows the user to enter an allowable range for one or more control parameters (cf. range data 24 in FIG. 2C). Alternatively or additionally, one or more allowable ranges may be predefined for the control device 20. Thereby, the adjustment of a candidate set value in step 403 may be confined to the allowable range for the related control parameter. The allowable ranges provide the advantage of enabling the set values to be tailored to be requirements of a clinic, the needs of an individual physician or the patient, etc.

The procedure 404B may be automatically terminated if steps 410-413 are repeated for longer than a timeout period. If the timeout period is exceeded, the method 400 may also inform the user, via the UI 17, of a need to modify the treatment target(s) and/or the control parameter(s) designated as modifiable (if defined) and/or the allowable range of one or more control parameters (if defined).

FIG. 5 is a plot of time-resolved values that have been calculated in step 404B for a PD session to be performed by a PDA 1. The bottom graph in FIG. 5 shows fluid volume in the peritoneal cavity as a function of time. As seen, the session comprises an initial drain phase followed by five fluid exchange cycles. The session is terminated by a drain phase. The middle graph in FIG. 5 shows concentration of glucose in the fluid within the peritoneal cavity as a function of time. The top graph is included to illustrate that it is also possible to calculate time-resolved values of conductivity for the fluid within the peritoneal cavity as a function of time, when time-resolved values of concentration for different solutes have been calculated in step 404B. Specifically, conductivity at the respective time step may be calculated by aggregating the contributions of the different solutes at the time step.

In some embodiments, the kinetic model used in the control method 400 is based on the well-known Three-Pore Model (TPM) of the peritoneum. The TPM is a transport model that assumes that the blood vessel wall of the peritoneum has three types of pores with different pore radii, enabling passive transport of molecules with different size properties. The smallest pore type, called Aquaporine-1 (AQP-1), is a water selective pore-structure. The AQP-1 pore enables passive transport of water, i.e. UF driven by osmotic force. A medium pore type permits transport of fluid and smaller solutes. The majority of protein transport over the membrane is enabled by a large pore type. Examples of TPM are found in WO2018/041760 and the article “Optimizing Automated Peritoneal Dialysis Using an Extended 3-pore Model”, by C. Öberg and B. Rippe, published in Kidney Int Rep., 2 (5): 943-951 (2017), which are both incorporated herein in their entirety by reference.

In some embodiments, the peritoneum transport model is configured to account for ion transport by electrostatic force across the peritoneum caused by differences in amounts of dissolved ions on opposite sides of the peritoneum and reflection of large charged solutes by the peritoneum. An example of such a mathematical transport model is given in Appendix A. A further example of incorporation of electrostatic force in a transport model is found in Chapter 17 (pp 33-36) of the publication “Analysis of Transvascular Transport Phenomena in the glomerular and peritoneal microcirculation”, by Öberg, Carl, (1 ed.), Lund: Lund University: Faculty of Medicine, ISBN 978-91-7619-372-3. It is currently believed that more accurate results are achieved by accounting for the electrostatic force. It may be noted that there are alternative and/or simpler techniques for accounting for the ion transport by electrostatic force, for example by use of so-called Donnan factors.

Equations representing the processes in FIG. 3 are presented in detail in Appendix A. While these equations are based on TPM, other kinetic models may be used instead, for example the Two-Pore Model, the Dual Barrier Membrane Model, or the Distributed Model, as readily understood by the skilled person.

Equations 1˜4 in Appendix A may be generalized into basic time-dependent governing functions for fluid flow through the peritoneum, Jᵥ(t), for the flow of solute i through the peritoneum, J; (t), for the temporal change in intraperitoneal volume, dVₚ/dt, and for the temporal change in concentration of solute i in the treatment fluid in the PC, dCₚᵢ/dt:

$${J}_{v} ( t ) = f 1 ( LpS , {V}_{p} ( t ) , {C}_{p 1 … N} ( t ) ) \\ {J}_{i} ( t ) = f 2 ( {V}_{p} ( t ) , {C}_{p 1 … N} ( t ) , {PS}_{1 … N} , {J}_{v} ( t ) ) \\ \frac{{dV}_{p} ( t )}{dt} = f 3 ( {J}_{v} ( t ) , {J}_{f} ( t ) , {J}_{d} ( t ) , {J}_{s} ( t ) ) \\ \frac{{dC}_{pi} ( t )}{dt} = f 4 ( {J}_{i} ( t ) , {J}_{v} ( t ) , {J}_{f} ( t ) , {V}_{p} ( t ) , {C}_{pi} ( t ) , {C}_{Ii} )$$

where C_{p1 . . . N} (t) designates the ensemble of concentration values for all included solutes at time t, Cₗᵢ designates the concentration of solute i in the fresh treatment fluid, and PS_{1 . . . N} designates the ensemble of PS values for all included solutes.

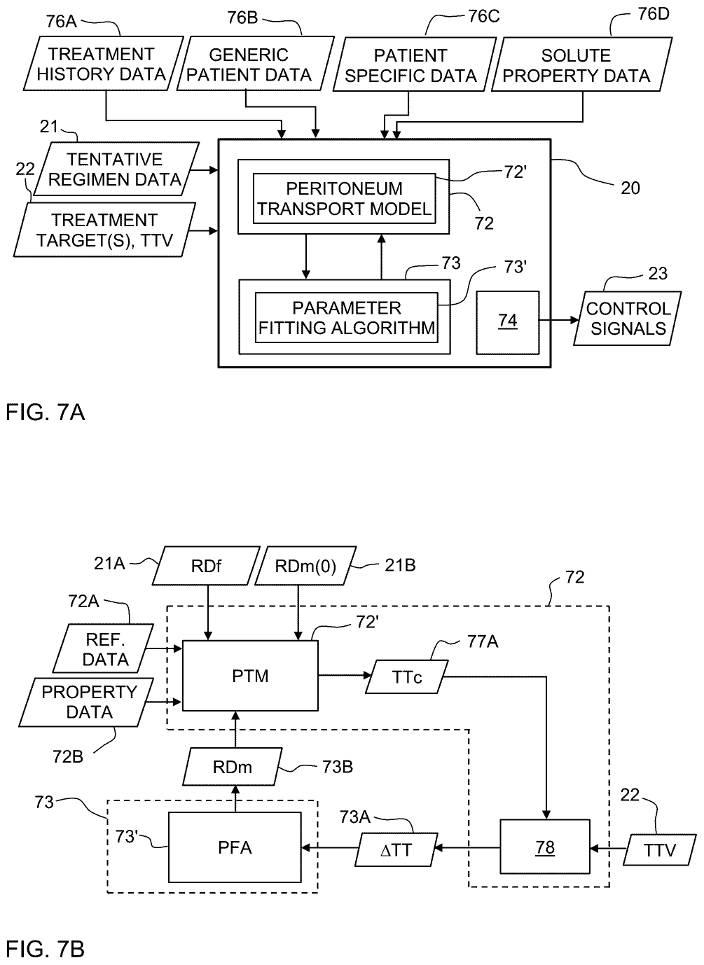

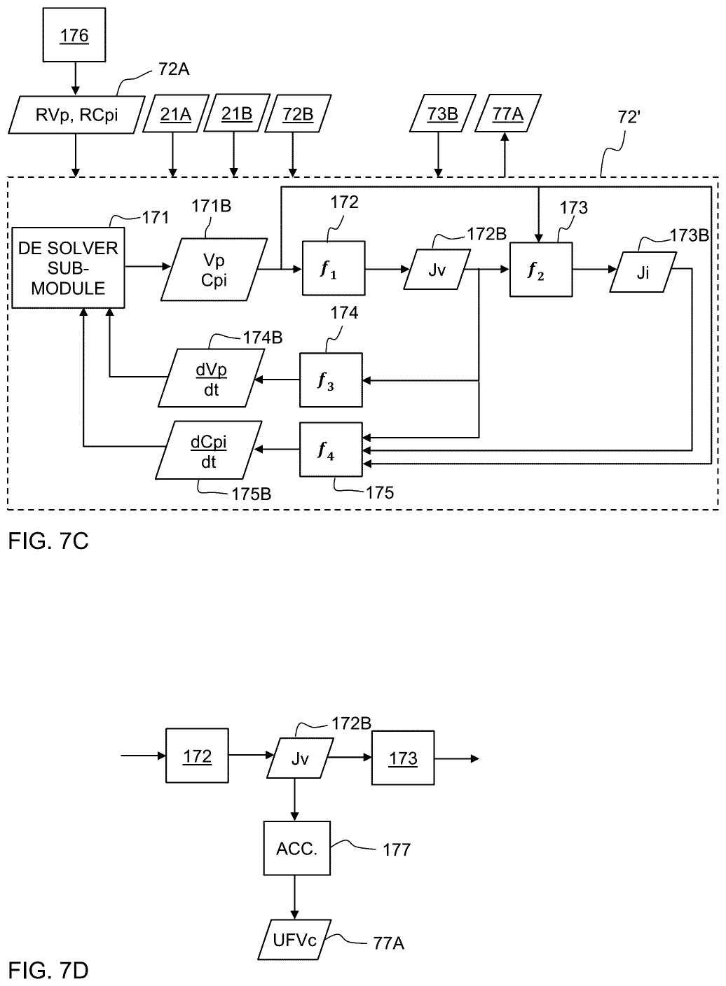

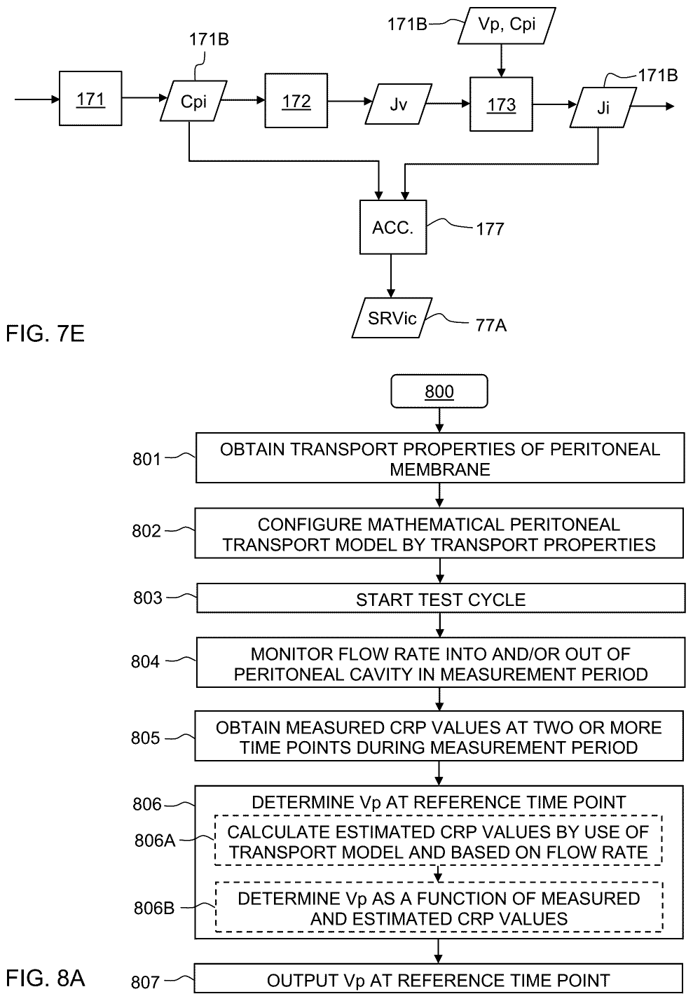

As seen, and understood from Appendix A, the governing functions have a complex and intermixed dependence on time-dependent variables shown in FIG. 3, as well as on the transport properties, LpS and PSi. The governing functions ƒ1, ƒ2, ƒ3, ƒ4 may be combined to define a peritoneum transport model, as will be exemplified further below with reference to FIGS. 7A-7C.

It may be noted that the above-mentioned scale factor, fCpw, is included in Cbi (cf. FIG. 3), which is used for calculation of both Jᵥ(t) and Jᵢ(t) (cf. Eq. 2 and Eq. 4 in Appendix A). Thus, the governing functions ƒ1 and ƒ2 will also depend on fCpw, if used.

It may also be noted from Appendix A that function ƒ2 may operate on PS_{i,m}, which designates a pore-type-specific value. In some embodiments, the PS values for large pores (PS_{i,3}) are set to generic values, for example given by the above-mentioned generic set, and the scaling factor fPS may be applied only to the PS values for small pores (PS_{i,2}). The PS value for a specific solute may then be computed by scaling the sum of the corresponding generic PS values for small and large pores by the scaling factor fPS. This simplification has been found to have little impact on the accuracy of the results. However, in other embodiments, the scaling factor fPS may be applied to the PS values for both small pores (PS_{i,2}) and large pores (PS_{i,3}). As a further simplification, the PS value for a specific solute may be computed by scaling the corresponding PSiG for small pores by the scaling factor fPS and thus omitting the contribution from large pores. This further simplification is at least applicable to small solutes, for example glucose or sodium, for which the contribution to the PSi from large pores may be effectively negligible.

Reverting to the control method 400 in FIG. 4A, step 403 of configuring the transport model may involve applying a known value of LpS in function ƒ1, and applying known values of PS_{1 . . . N} in function ƒ2.

It may be noted that, given the notations in FIG. 3, the treatment target UFV is given by an aggregation of (Jᵥ(t)−L) for the duration of the session, and that the treatment target SRVi is given by an aggregation of Jᵢ(t) for the duration of the session. Further, the control parameters in the regimen definition 21′ of FIG. 2C are represented by variables in the functions ƒ1-ƒ4. The fluid composition (FC) is given by Cₗᵢ, the fill volume (Vf) is embedded in J_{f}(t), and the number of cycles (NoC), the cycle frequency (CF), the treatment time (TT) and the modality (TM) are all embedded in J_{f}(t) and J_{d}(t). Thereby, it is possible to calculate set values for the control parameters so as to achieve one or more treatment targets during a session based on a transport model given by the functions ƒ1-ƒ4.

FIG. 7A is a block diagram of an example control device 20 for operating a PD arrangement, for example PDA 1 in FIG. 1. The control device 20 is configured to perform the setup procedure 400A in accordance with FIGS. 4A-4B. In the illustrated example, the control device 20 is configured to receive input data comprising tentative regimen data 21, one or more treatment targets (TTV) 22, treatment history data 76A, generic patient data 76B, patient specific data 76C, and solute property data 76D. The control device 20 comprises a first computation module 72 and a second computation module 73, which are cooperatively operated to determine a regimen comprising set values of control parameters. The set values are provided to a signal generator 74, which is configured to generate and output corresponding control signals 23 for the DMU 2/2a in the PDA 1 (cf. FIG. 2C). The first computation module 72 defines or comprises the transport model (PTM) 72′. For example, the PTM 72′ may be an implementation of the governing functions ƒ1-ƒ4. The first computation module 72 is configured to operate the PTM 72′ on at least part of the input data to calculate candidate set values (cf. step 410). The second computation module 73 defines or comprises a parameter fitting algorithm (PFA) 73′, which is configured to operate on the candidate set values from the first computation module 72 to calculate a current target value (cf. step 411). The first and second computation modules 72, 73 are configured to alternately generate the candidate set values and the current target value until the PFA 73′ finds that the current target value fulfils a convergence criterion, or a time limit expires (cf. step 412). The control device 20 then operates the signal generator 74 to generate and output the control signals 23. The PFA 73′ may be any algorithm (“optimization procedure”) capable of solving non-linear optimization problems and enabling fitting of experimental data to simulated data. Such algorithms include any non-linear programming (NLP) algorithm such as algorithms for least-squares minimization. The results presented herein has been generated by use of the standard function Lsqnonlin in MATLAB.

Looking more in detail at the input data presented in FIG. 7A, the regimen data 21 defines a tentative regimen, which may be at least partly entered by the user. In some embodiments, the tentative regimen comprises set values of fixed control parameters and tentative set values of modifiable control parameters. The treatment history data 76A defines dialysis treatment that has been performed on the patient in a preceding time period, for example within 12-48 hours of the PD session. For example the treatment history data 76A may include regimen data for one or more fluid exchange cycles performed on the patient in a preceding time period. The generic patient data 76B may include the above-mentioned generic set of PSi values, as well as generic values of plasma concentrations of solutes (cf. Cbi in FIG. 3). In some embodiments, all solutes that are expected to be present in blood or treatment fluid in a concentration of at least about 0.5 mmol/L are included in the PTM 72′ and thus also in the generic patient data 76B. The generic values may be given as population averaged values. It is understood that the generic patient data 76B may also include other parameter values used by the PTM 72′, such as @m (see Appendix A). The patient specific data 76C may include any known or estimated property of the patient of relevance to the calculations by the control device 20, such as values of LpS and PSi. The patient specific data 76C may also include concentrations of one or more solutes in the patient's blood, or a previously determined value of Vres, or fCpw (cf. FIG. 3). The solute property data 76D may include any known property data of the solutes that are included in the governing functions used by the PTM 72′. In the example of Appendix A, the solute property data 76D may include one or more of osmotic coefficients (φᵢ), charges of the solutes (zᵢ), etc.

The input data shown in FIG. 7A and described above is not intended to be limiting and is only provided as an example. It is conceivable that additional input data is used and/or that one or more of the datasets 76A-76D are omitted. For example, the patient specific data 76C may not be available. In another example, all or part of the solute property data 76D may be integrated into the PTM 72′. Further, the treatment history data 76A may be omitted altogether.

FIG. 7B is a block diagram of a more detailed example of the first and second computation modules 72, 73. The PTM 72′ is configured to receive target data 22 comprising the treatment target(s), TTV (cf. step 401), property data 72B comprising values of the transport properties LpS and PSi (cf. step 402), and reference data 72A comprising the reference values of fluid volume and solute concentration (cf. step 404A). The property data 72B may be included in the patient specific data 76C (FIG. 7A) or entered by the user. In the example of FIG. 7B, the PTM 72′ is also configured to receive fixed regimen data (RDf) 21A, which defines values of all fixed control parameters (if any), and initial regimen data 21B (RDm(0)), which defines initial values of all modifiable control parameters (if any). The first and second regimen data 21A, 21B may be included in the tentative regimen data 21 in FIG. 7A.

As shown in FIG. 7B, the PTM 72′ is operable to calculate a current target value (TTc) 77A of the treatment parameter(s) based on the reference data 72A, the property data 72B, as well as the regimen data 21A, 21B (cf. steps 410-411). The current target value TTc 77A is received by a subtraction module 78, which is configured to compute the difference between TTV and TTc (cf. step 412), resulting in error data (ATT) 73A. If TTV and TTc comprise more than one treatment parameter, a difference is calculated for each treatment parameter and included in the error data 73A. The error data 73A is received by the second computation module 73. The PFA 73′ is configured to operate on the error data 73A to generate candidate set values of the control parameters (cf. step 413) as represented by RDm 73B in FIG. 7B. In the example of FIG. 7B, in view of RDf 21A, the PFA′ 73 refrains from modifying the fixed control parameters and generates RDm 73B to include candidate set values of modifiable control parameters only. Upon receiving RDm 73B, the PTM 72′ is configured to operate on RDm 73B and RDf 21A, as well as the reference data 72A and the property data 72B, to calculate a new current target value TTc 77A. As understood from the foregoing, the calculations and flow of data may continue until the PFA 73′ finds that a convergence criterion is fulfilled, for example that the error data 73A is small enough. The control device 20 in FIG. 7B may be seen to represent a feedback control system in which the first computation module 72 corresponds to the system to be controlled, TTV 22 corresponds to a set value, TTc 77A corresponds to an actual value, and the PFA 73′ corresponds to the controller.

In some embodiments, the property data 72B is presumed to be time invariant (constant) during the PD session, which may facilitate the calculations. However, it is also possible for one or more transport properties in the property data 72B to be time varying by including a predefined time dependence for the respective property. For example, it is previously known to model a declining time dependence of PSi and/or LpS during PD, for example as described in the article “Diffusive Mass Transfer Coefficients Are Not Constant During a Single Exchange In Continuous Ambulatory Peritoneal Dialysis”, by Waniewski et al, published in ASAIO J 1996; 42: M518-523, which is incorporated herein in its entirety by reference.

The subtraction module 78 need not be included in the first computation module 72, as shown in FIG. 7B, but may instead be included in the second computation module 73 or in a separate (third) computation module.

FIG. 7C is a block diagram of a more detailed example of the PTM 72′ in FIG. 7B. Generally, the PTM 72′ is configured to implement the governing functions ƒ1-ƒ4. In the illustrated example, the PTM 72′ comprises a differential equation solver (DES) sub-module 171 which is configured to operate on a value of the derivative (“temporal change”) of a variable at one or more previous time steps to generate the value of variable at a current time step. For example, the DES sub-module 171 may implement any known regression method for obtaining numerical solutions to differential equations, such as a linear multistep method, a Runge-Kutta method, or a general linear method (GLM). The results presented herein have been generated by implementing the PTM 12′ on a conventional ODE (Ordinary Differential Equation) solver, specifically the ode45 function in MATLAB.

In the illustrated example, the PTM 72′ is configured to generate a time series of values of the intraperitoneal volume, Vp, and a corresponding time series of values of the concentrations of solutes, Cpi, in the fluid in the PC. To this end, the PTM 72′ further comprises governing sub-modules 172-175, which implement a respective governing function ƒ1-ƒ4. During operation, the DES sub-module 171 generates a dataset 171B comprising Vp, Cpi for the current time step based on datasets 174B, 175B comprising the derivatives of Vp and Cpi for the preceding time step. The governing sub-module 172 operates on Vp, Cpi for the current time step to generate Jv for the current time step. The governing sub-module 173 operates on Jv, Vp, Cpi for the current time step to generate Ji for the current time step. The governing sub-module 174 operates on Jv for the current time step to generate the derivative of Vp for the current time step. The governing sub-module 175 operates on Jv, Ji, Cpi, Vp to generate the derivative of Cpi for the current time step. It is realized that by operating the FTM 72′ from a start time (t=0) to an end time, a respective time series of values of Vp and Cpi are generated. The PTM 72′ is configured to generate the respective time series for the treatment time period, which is given by the control parameter TT (FIG. 2C) in the regimen data 21A or 21B.

As understood from the foregoing and indicated in FIG. 7C, the PTM 72′ operates on reference data 72A, regimen data 21A, 21B and property data 72B when generating the two time series. As stated, the control parameter TT in the regimen data defines the treatment time period. As noted above, the regimen data also defines other variables in the governing functions ƒ1-ƒ4 and is at least used by sub-modules 174, 175. Likewise, the property data 72B is at least used by sub-modules 172, 173.

The DES sub-module 171 derives the intraperitoneal volume at the reference time point, RVp, and the concentrations of the solutes in the PC at the reference time point, RCpi, from the reference data 72A. The reference time point may or may not be the start time for the session. If the reference time point equals the start time, RVp and RCpi may be used as initial seed values in the dataset 171B when the PTM 172′ starts calculating Vp and Cpi. If the reference time point differs from the start time, the operation of the PTM 172′ is slightly modified, as readily understood by the skilled person.

The DES sub-module 171 may retrieve the reference data 72A from memory. As noted above, RVp and RCpi may have been determined from preceding measurements or estimated by calculations. In the example of FIG. 7C, the reference data 72A is provided by a reference data module 176. In some embodiments, the reference data module 176 is configured to calculate RVp and/or RCpi from the generic patient data 76B or the patient specific data 76C (FIG. 7A). The use of generic patient data 76B may lower the accuracy of the resulting set values, if RCpi or RVp is significantly inaccurate. In some embodiments, to mitigate this potential problem, the reference data module 176 is configured to run a simulation based upon the information in the treatment history data 76A (FIG. 7A) about dialysis treatment performed in a preceding time period. The simulation may be performed by use of the governing functions ƒ1-ƒ4 in view of these regimen(s). Initial values of the solute concentrations for this simulation may be taken as plasma water concentrations, optionally slightly modified from plasma water by, for example, reducing the content of large solutes and/or by adjusting sodium and chloride according to Donnan equilibrium. Alternatively, the reference data module 176 may be configured to set RCpi equal to the plasma water concentration, optionally while applying a reduction factor for large solutes such as albumin. In another alternative, the reference data module 176 may be configured to set RCpi equal to the concentration of the respective solute in the fresh treatment fluid.

When the time series of values for Vp and Cpi have been generated, the PTM 72′ calculates the current target value TTc as a function of at least one of the time series. The calculated TTc is then output for receipt by the subtraction module 78 (FIG. 7B). When the PFA 73′ has calculated RDm 73B based on ATTc 73A, as produced by the subtraction module 78, the PTM 72′ is again operated to generate a respective time series of values of Vp and Cpi. Here, the PTM 72′ may again use the reference data 72A, the property data 72B and RDf 21A.

FIG. 7D shows an example structure in the PTM 72′ for calculating a current target value 77A of fluid removal, designated as UFVc. The structure comprises an accumulation module 177, which is configured to receive and accumulate the momentary values of Jv that are generated by governing sub-module 172. Functionally, the accumulation module 177 performs an integration of Jv(t) (FIG. 3) over the treatment time period, resulting in a total water volume. The accumulation module 177 may also be configured to calculate an accumulated lymphatic water absorption for the treatment time period and subtract the accumulated lymphatic water absorption from the total water volume, resulting in a net water volume. If the lymphatic flow (L in FIG. 3) is assumed to be constant, the accumulated lymphatic water absorption may be calculated as the product of treatment time and lymphatic flow. Depending on implementation, UFVc may be given as the total water volume or the net water volume.

FIG. 7E shows an example structure in the PTM 72′ for calculating a current target value 77A of solute removal from the patient during the session, designated as SRVic. The structure comprises an accumulation module 177, which is configured to receive and accumulate the momentary values of Ji that are generated by governing sub-module 173. Functionally, the accumulation module 177 performs an integration of Ji(t) (FIG. 3) over the treatment time period, resulting in the total amount of solute i that has passed the peritoneal membrane into the PC (“total solute transport”). In the illustrated example, the accumulation module 177 is also configured to receive and process the momentary values of Cpi that are generated by governing sub-module 171.

Functionally, the accumulation module 177 performs an integration of L·Cpi(t) (FIG. 3) over the treatment time period, resulting in an accumulated lymphatic solute absorption. The current target value, SRVic, is calculated by subtracting the accumulated lymphatic solute absorption from the total solute transport. In a variant, not shown, SRVic may instead be given as the total solute transport.

The Applicant has found that the transport model may also be used to validate one or more of the transport properties used in the transport model, and optionally to validate the performance of the control method 400, by use of CRP values that are measured for the fluid in the PC during the session. Thus, in the embodiment of FIG. 4A, the control method 400 comprises a validation procedure 400B, which may be performed during or after the PD session that is initiated by the setup procedure 400A. In the example of FIG. 4A, the validation procedure 400B comprises steps 406-409.

In step 406, first validation data (“first data”) is obtained. The first validation data comprises a measured CRP value at one or more time points during the session. In FIG. 1, the control device 20 obtains the measured CRP value(s) from the CRP sensor 15. In one example, a CRP value may be measured during a drain phase, by the control device 20 directing the effluent through the CRP sensor 15. In another example, a CRP value may be measured during the dwell phase by the PDA 1 being operated to draw a small sample of fluid out of the PC via the patient line 7 and directing the fluid sample through the CRP sensor 15. Such a fluid sample is represented by Js(t) in FIG. 3. The respective measured CRP value in the first validation data may be given by a single measurement value from the CRP sensor 15, or as a time-average of a plurality of measurement values.

In step 407, second validation data (“second data”) is obtained. The second validation data is representative of the fluid flow into and out of the PC through the access 7′ (FIG. 1) during the session. For example, the control device 20 may obtain the second validation data from the flow meter 13 or based on volumetric monitoring. Alternatively or additionally, the second validation data may be given by the set values that have been applied by the control device 20 to perform the session up to the time of the validation, for example the set values that were calculated in step 404 of the setup procedure 400A. The second validation data designates fluid flow as a function of time during the session. In some embodiments, the second validation data comprises values of fill volume, drain volume and sample volume (if relevant) for the respective cycle, as well as start and end times for phases of the respective cycle. Such phases include fill phase, drain phase, and any sampling phase for extracting a fluid sample from the PC, and the values of fill volume, drain volume, and sample volume designate an accumulated volume for the respective phase. In other embodiments, the second validation data comprises a time series of momentary flow rate values for the fluid flow through the access 7′ during the session.

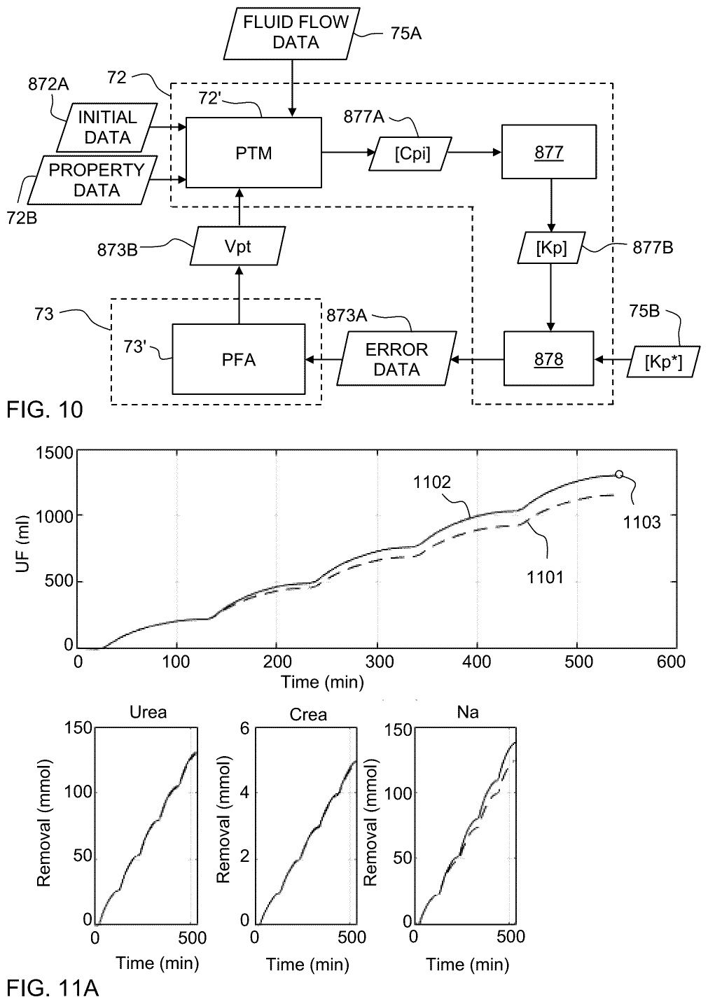

In step 408, at least one of the transport properties is recalculated by use of the transport model. Thus, step 408 results in at least one updated transport property of the peritoneum. The recalculation is based on the first and second validation data and typically results in a value of at least one of LpS or PSi. Step 408 may be performed at any time during the session, at completion of the session, or at any time after completion of the session. Reverting to the governing functions ƒ1-ƒ4, the skilled person understands that LpS and/or PSi may be calculated if other variables are known or estimated. The flow-related variables Jf(t), Jd(t), Js(t) are known from the second validation data. It can be shown that, for calculating one updated transport property, it is sufficient for the first validation data to include measured CRP values at two different time points. A larger number of measured CRP values will improve accuracy (confidence) and/or enable calculation of another updated transport property. The measured CRP values may be taken in at least two different phases during the PD session, for example during a drain phase and a consecutive dwell phase, or during a dwell phase and a consecutive drain phase. The calculation of updated transport properties will be further explained below in conjunction with FIG. 10.

In step 409, one or more dedicated actions are performed based on the one or more updated transport properties calculated in step 408. Examples of such dedicated actions are shown in FIGS. 4C-4E.

In the example of FIG. 4C, the dedicated action is a step 409A of displaying and/or storing the calculated value of the respective updated transport property. In the example of FIG. 1, the calculated value may be stored in the memory 18 (local or external) and/or presented on the UI 17, for example as part of an electronic medical record (EMR) or in a remote patient monitoring system. It is understood that the calculated value from step 408, being given by first and second validation data for an on-going session, is likely to represent the patient's peritoneum more accurately than the previous value that was entered in step 402. Thus, by step 409A, the caretaker is given a better understanding of the current status of the patient's peritoneum. The calculated value may be used for any suitable purpose, including to improve the performance of future PD treatment of the same patient. For example, the calculated value may be entered by the caretaker or retrieved from the memory in step 402 during a future execution of the control method 400.

In the example of FIG. 4D, the dedicated action comprises steps 409B, 409C, 409D and is performed during on-going PD treatment. In step 409B, the transport model is reconfigured with the respective updated transport property. Step 409B may be performed by analogy with step 403, by replacing a current value of the respective transport property by the calculated value from step 408. For example, step 403 may involve applying a recalculated value of LpS in function ƒ1 and/or applying recalculated values of PS_{1 . . . N} in function ƒ2. In step 409C, set values of the control parameters are recalculated. Step 409C may comprise a calculation procedure, in which the reconfigured transport model is evaluated to determine updated set values to achieve the treatment target for the PD session (TTV from step 401). The calculation procedure of step 409C is performed to account for the second validation data from step 407 and possibly the first validation data from step 406. In one implementation of the calculation procedure, an intermediate value of the treatment parameter(s) is calculated based on the second validation data. The intermediate value represents how much of the treatment target that is achieved at the current time point. Then, an updated treatment target is calculated by subtracting the intermediate value from the treatment target. The calculation procedure then performs step 404, by use of the reconfigured transport model from step 409B, to determine the updated set values for the PDA 1 to achieve the updated treatment target. The reference data 72A may also be updated based on the first and second validation data, in accordance with method 800 (FIGS. 8A-8B below). In step 409D, updated control signals for the PDA 1 are generated based on the updated set values, by analogy with step 405. The PDA 1 is thereby operated in accordance with the updated set values to achieve the TTV entered in step 401 for the session as a whole.

The dedicated action in FIG. 4D will improve the performance of the PD treatment in terms of its ability to reach the treatment target, by automatically accounting for the effect of any difference between the value of respective transport property as originally entered in step 402 and the calculated value in step 408.

In the example of FIG. 4E, the dedicated action comprises steps 409B, 409F and 409G and is performed during on-going PD treatment. In step 409B, as described above, the transport model is reconfigured with the respective updated transport property. In step 409F, a final value of the treatment parameter(s) at completion of the ongoing PD session is calculated by use of the updated transport model and based on the second validation data from step 407. The final value thus represents the actual TTV that will be achieved by the on-going session (“actual value at therapy end”). The final value may be calculated to represent at least one of fluid removal or solute removal. In step 409G, the final value of fluid removal and/or solute removal is stored and/or presented to the user. In the example of FIG. 1, the final value may be stored in the memory 18 (local or external) and/or presented on the UI 17.

It is realized that the final value designates the actual change in the patient's physiological status as a result of the session. The final value allows the physician to verify the effectiveness of the session. The final value may also be used by the physician when determining the treatment target for a forthcoming session. In a further variant, the control device 20 may comprise a calculation function for automatically calculating a treatment target for a forthcoming session based on the final value of the preceding session, for example to achieve an overall treatment target for a plurality of sessions. The final value may be manually input to the calculation function by the caretaker via the UI 17, or be automatically retrieved by the control device 20 from the memory 18.

It is to be noted that one or more of the dedicated actions represented by FIGS. 4C-4E may be combined.

Another dedicated action, not shown in the drawings, is to perform step 409B to reconfigure the transport model with the respective updated transport property from step 408, to thereby prepare a reconfigured (updated) transport model for use the next time the control method 400 is to be executed. Such a dedicated action presumes that one and the same patient is treated by the PDA and may, for example, be applicable to home dialysis.

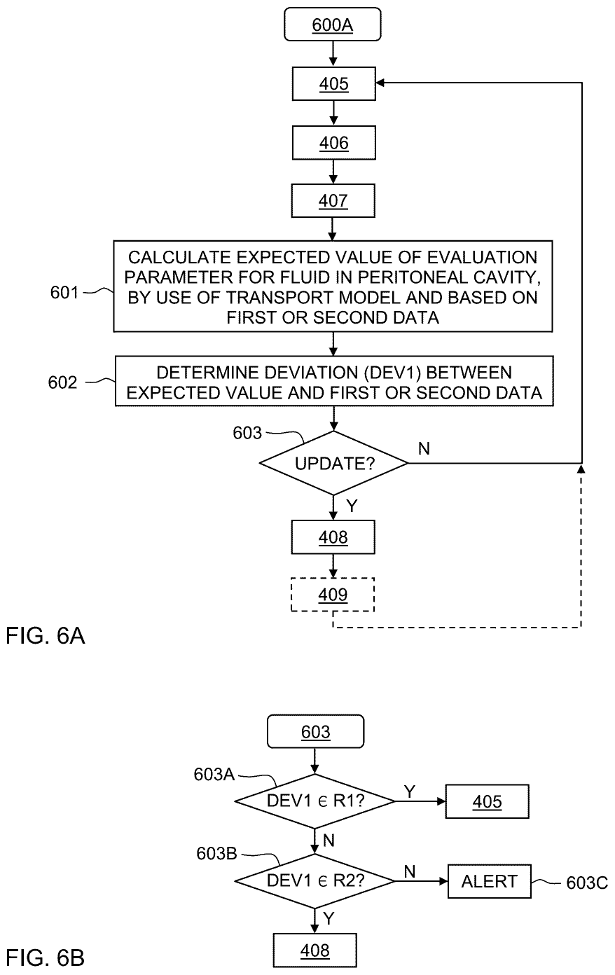

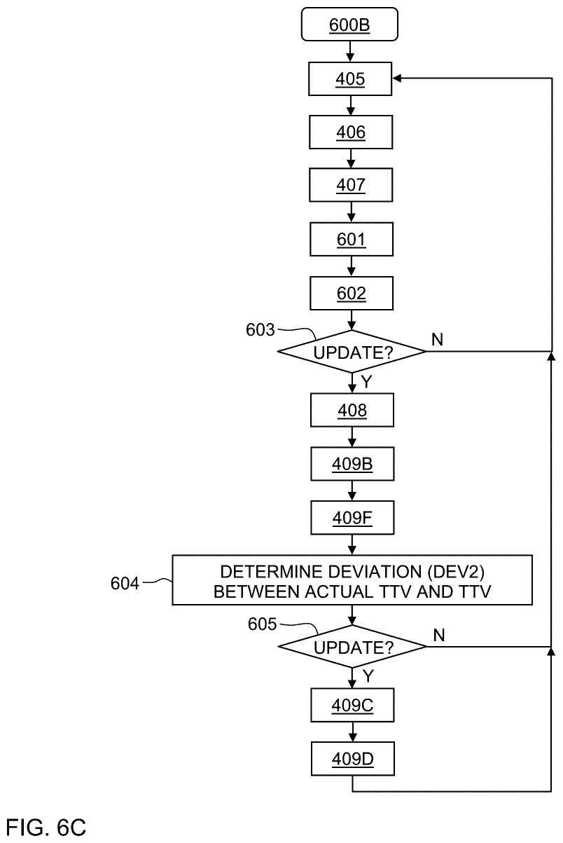

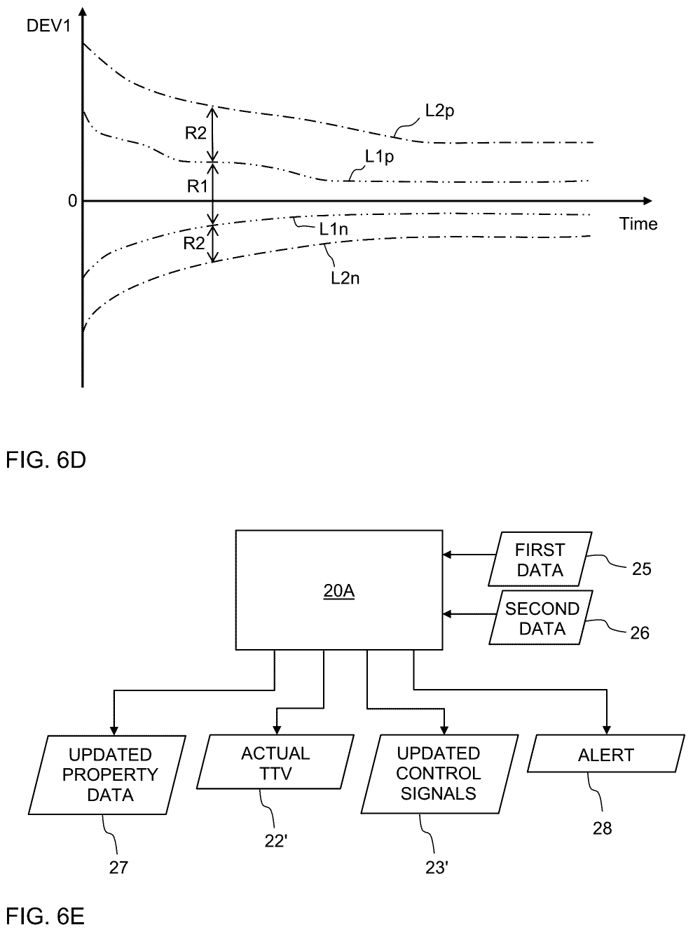

FIG. 6A is a flowchart of an example method 600A, which comprises an embodiment of the validation procedure 400B. The method 600A is performed while the PDA 1 is operated by step 405 in accordance with the set values determined by the setup procedure 400A.

The method 600A comprises, in accordance with the validation procedure 400B, a step 406 of obtaining first validation data, which includes a measured CRP value at one or more time points during the session, and a step 407 of obtaining second validation data. in step 601, a respective expected CRP value is calculated at the time point of the respective measured CRP value. The expected CRP value is calculated by use of the transport model from step 403 (FIG. 4A) and based on the second validation data. Reverting to FIG. 7C, it is realized that the PTM 72′ is operable to generate a time series of Cpi values if all required input data to the PTM 72′ is known. Since the PDA 1 is operated in accordance with the set values determined by the setup procedure 400A, all required input data is inherently known. Thus, given the transport model, it is a straight-forward task to calculate a Cpi value of a respective solute at any given time point during an on-going session. Depending on CRP, step 603 may further comprise a conversion of one or more calculated Cpi values at a time point into the expected CRP value. For example, if CRP values represent conductivity, Cpi values need to be converted into conductivity. The conversion may be performed as discussed above in relation to FIG. 5.

In steps 602-603, the measured and expected CRP values are evaluated in relation to a deviation criterion. In the illustrated embodiment, an evaluation parameter (first deviation, DEV1) representing the difference between corresponding measured and expected values is calculated in step 602. If measured CRP values are available at plural time points, DEV1 may be an aggregation of such differences. In step 603, DEV1 is evaluated in relation to the deviation criterion. If DEV1 is found to represent a sufficiently large difference (deviation), step 603 proceeds to step 408, in which at least one transport property is recalculated (updated). Thus, the method 600A selectively, based on DEV1, recalculates at least one transport property. If DEV1 is found to be sufficiently small in step 603, the method 600A returns to step 405 and the control unit 20 continues to operate the PDA 1 in accordance with the set values that were determined by the setup procedure 400A. As indicated by dashed lines, step 408 may be followed by an action step 409, for example in accordance with any one of FIGS. 4C-4E and/or the method 600A may return to step 405 after step 408 or step 409.

The control device 20 may be configured to repeatedly perform the sequence of steps 406, 407, 601, 602, 603 during a PD session, for example at a fixed time interval or at otherwise selected time points.

The combination of steps 601-603 is implemented to trigger the recalculation of at least one transport property whenever a sufficient deviation is detected between modeled conditions and actual conditions. By properly defining the deviation condition, it is possible to avoid unnecessary recalculation of transport properties by step 408 as well as to avoid that unnecessary actions are taken by step 409. Thus, the embodiment in FIG. 6A may serve to reduce the processing load and energy consumption of the control device 20.

In the foregoing example, the modeled and actual conditions are represented by CRP value(s). Thus, the expected value is calculated by use of the transport model and the first validation data (step 601), and the deviation is determined between the expected value and a corresponding value given by the second validation data (step 602). This is believed to provide a simple and robust detection of a deviation between the modeled and actual conditions. In an alternative, the expected value is calculated by use of the transport model and the second validation data (step 601), and the deviation is determined between the expected value and a corresponding value given by the first validation data (step 602). For example, the expected value may be an expected drain volume for a drain phase (cf. Vd in FIG. 2A), and the deviation may be determined between the expected drain volume and an actual drain volume given by the second validation data.

FIG. 6B is a flow chart of a procedure that may be part of the evaluation step 603 in FIG. 6A. The procedure in FIG. 6B involves comparing DEV1 to a plurality of ranges. An example of such ranges is shown in FIG. 6D. In the illustrated example, the ranges are defined by a first positive limit L1p, a second positive limit Lp2, a first negative limit Lin, and a second negative limit L2n, where L2n<Lin and L2p>L1p. Specifically, a first range R1 is defined between Lin and L1p, and two second ranges R2 are defined between L2n and Lin, and between Lp1 and Lp2, respectively. Thus, the first range R1 is located around and closer to DEV1=0 than the second ranges R2. It may be noted that the absolute values of L2n, L2p may or may not be identical. Likewise, the absolute values of L1n, L1p may or may not be identical.

In the example of FIG. 6D, the limits of the ranges R1, R2 varies over time. Specifically, the extent (width) of R1 decreases monotonically with time from start to end of the PD session. Such a narrowing of R1 may be beneficial when the transport model is repeatedly updated during a PD session, subject to the outcome of step 603, for example if step 409 in FIG. 6A comprises step 409B. The underlying rationale is that the accuracy of the transport model should be improved by every update, causing DEV1 to become smaller over time. By gradually narrowing the range R1, the method 600A is more likely to be continually triggered (in step 603) to improve the transport model by updating the transport property/properties (step 408) and by reconfiguring the transport model by the updated transport property/properties (step 409B). The outer limits (L2p, L2n) of the ranges R2 may be gradually converging to improve the sensitivity of the method 600A to malfunctions of the PDA. The extent (width) of the respective range R2 may remain the same over time, or be gradually narrowed, depending on implementation. It is realized that FIG. 6D is a non-limiting example. Generally, the control device 20 may be configured to narrow the range R1 (difference between L1p and L1n) over time by modifying at least one of L1p, L1n. Similarly, the control device 20 may be configured to decrease the difference between L2n, L2p over time by modifying at least one of L2n, L2p.

Reverting to FIG. 6B, the illustrated procedure comprises first and second evaluation steps 603A, 603B. In step 603A, DEV1 is evaluated in relation to the first range R1. If DEV1 falls within R1, step 603A proceeds to step 405, which means that the method 600A refrains from calculating any updated transport property. Thus, R1 is defined to include acceptable deviations between the modeled and actual conditions as given by steps 601-602 (FIG. 6A). If DEV1 falls outside of R1, step 603A proceeds to step 603B, in which DEV1 is evaluated in relation to the second ranges R2. If DEV1 falls within one of the second ranges R2, step 603B proceeds to step 408, which means that at least one transport property is recalculated (updated). On the other hand, if DEV1 falls outside of the second ranges R2, step 603B proceeds to step 603C in which an alert for the user is generated, for example via the UI 17 (FIG. 1). The alert may instruct the user to check the PDA 1, for example that it is properly installed, that tubing is not kinked, etc. In some embodiments, step 603C may also cause the control device 20 to stop the PDA or even terminate the PD session. It is realized that the limits L2n and L2p are set to define the minimum and maximum deviations that are allowed to occur as a result of inaccuracies in the transport model, so that deviations beyond L2n and L2p are likely to be the result of a malfunction of the PDA. The procedure in FIG. 6B thus reduces the processing load of the control device 20 while also performing a safety function for detecting operational errors or malfunctions of the PDA.

It may be noted that the procedure in FIG. 6B refrains from performing step 408 when DEV1 causes the alert to be generated by step 603C. This saves processing resources and also avoids the risk that a potentially erroneous value of the transport property is calculated based on the first and second validation data from steps 406, 407. However, in a variant, step 408 is performed in conjunction with step 603C.

FIG. 6C is a flow chart of an example method 600B, which corresponds to the method 600A of FIG. 6A and is implemented to selectively generate updated control signals for the PDA. Like the method 600A, the method 600B is performed while the PDA 1 is operated by step 405 in accordance with current set values. In the embodiment of FIG. 6C, step 408 is followed by a step 409B, in which the transport model is reconfigured with the respective updated transport property, and a step 409F, in which the actual TTV for the on-going session is calculated by use of the updated transport model and based the second validation data from step 407. Steps 409B, 409F may be performed as described hereinabove. Then, in step 604, a second evaluation parameter (second deviation, DEV2) is calculated. DEV2 represents the difference between corresponding the actual TTV and the TTV entered in step 401 (FIG. 4A). In step 605, DEV2 is evaluated in relation to a second deviation criterion. If DEV2 is found to be sufficiently small in step 605, the method 600B returns to step 405 and the control unit 20 continues to operate the PDA 1 in accordance with the current set values. If DEV2 is found to represent a sufficiently large difference, step 605 proceeds to step 409C, in which updated set values are determined to achieve the treatment target. Then, in step 409D, updated control signals for the PDA 1 are generated based on the updated set values. The method 600B returns to step 405 and the control unit 20 now operates the PDA 1 in accordance with the set values determined by step 409C. Steps 409C and 409D may be performed as described hereinabove. It is realized that the method 600B selectively, based on DEV2, evaluates the updated transport model to determine updated set values, and generates updated control signals for the PDA in correspondence with the updated set values. The method 600B provides a processing efficient way of ensuring that the TTV is achieved during a PD session. In a variant, steps 409F, 604 and 605 are omitted.

The Applicant has realized that the transport model may be used for estimating the reference value RVp, which may be used in step 404 of the method 400. In some embodiments, the intraperitoneal volume at the end of a preceding PD session is calculated and this final Vp is stored in memory (cf. 18 in FIG. 1). When set values are to be calculated in accordance with step 404 of the method 400 (FIG. 4A), the final Vp is retrieved from memory, and RVp is set equal to, or based on, the final Vp. However, it may be desirable to obtain a current value of RVp. This need is addressed by an example calculation method 800 depicted in FIG. 8A. The method 800 may be performed by the control device 20.

In step 801, transport properties of the peritoneal membrane are obtained, in correspondence with step 402 (FIG. 4A). In step 802, the transport model is configured by the transport properties obtained in step 801, in correspondence with step 403 (FIG. 4A). In step 803, the PDA 1 is operated to start a test cycle comprising, in sequence, a drain phase and a fill phase. Step 803 may be performed in accordance with a predefined test regimen, which defines the composition of the treatment fluid, the drain volume, the fill volume, and the duration of the sequence. In step 804, the fluid flow into and/or out of the PC via the access 7′ is monitored during a measurement time period (“test period”) that includes the test cycle. For example, the fluid flow may be monitored by the flow meter 13 or by volumetric monitoring. By step 804, the control device 20 obtains first measurement data, which is indicative of fluid flow through the access 7′ during the test period. By analogy with the second validation data (step 407 in FIG. 4A), the first measurement data may comprise values of fill volume, drain volume and sample volume for the test cycle, as well as start and end times for phases of the test cycle. In a variant, the first measurement data may comprise a time series of momentary flow rate values for the fluid flow through the access 7′ during the test period.

In step 805, second measurement data is obtained. The second measurement data comprises CRP values (“data samples”) of fluid in the PC at two or more time points (“measurement times”) during the test period. In the example of FIG. 1, the CRP values are measured by the CRP sensor 15. In step 806, the fluid volume in the PC at a reference time point is calculated by use of the transport model, configured in accordance with step 802, and based on the first and second measurement data. The calculated fluid volume is the RVp. In step 807, the calculated RVp is output, for example for use in the method 400. The reference time point may be any time point during the test cycle or a later time point. To facilitate calculations in the method 400, the reference time point may be selected to correspond to the end of a drain phase, so that RVp represents the residual volume, Vres.

As indicated by dashed lines in FIG. 8A, step 806 may comprise steps 806A, 806B. In step 806A, estimated CRP values at the measurement times are calculated by use of the transport model and based on the first measurement data. The estimated CRP values are determined from solute concentration values (Cpi) that are generated by the transport model. As noted above, depending on CRP, an estimated CRP value may be directly given by a solution concentration value or calculated by a conversion operation, for example from solute concentration value(s) to conductivity. In step 806B, the fluid volume in the PC at the reference time point is determined as a function of the measured CRP values and the estimated CRP values.

FIG. 8B is a flowchart of an example procedure that may be part of step 806 in the method 800. In step 810, the time-resolved values of fluid volume (Vp) and solute concentration (Cpi) in the PC are calculated by use of the transport model and based on the first measurement data and based on candidate values of Vp and Cpi at a selected time point. The selected time point may be any time point during the test period. Calculations may be facilitated if the selected time point coincides with the start of the test period (t=0). In step 811, estimated CRP values at the measurement times are determined based on the time-resolved values of solute concentration. Step 811 may be performed by analogy with step 601. In step 812, the estimated CRP values and the measured CRP values are evaluated in relation to a convergence criterion. The convergence criterion may be fulfilled if the (aggregated) difference between the estimated and measured CRP values is below a predefined limit. If the convergence criterion is not fulfilled, step 813 is performed. In step 813, the candidate Vp value is modified (updated) in accordance with an optimization procedure. The calculation in step 810 is then repeated for the updated candidate Vp value. Steps 810-813 are thus repeated until the convergence criterion is fulfilled, or a time limit is exceeded. When the convergence criterion is fulfilled, the procedure proceeds to step 814, in which RVp is determined. RVp may be given by the fluid volume at the reference time point in the time-resolved values of fluid volume calculated by step 810, for example in the last execution of step 810.

FIG. 10 is a block diagram of an example structure for implementing the method 800 according to FIGS. 8A-8B, for example in the control device 20. For brevity, the description will not be repeated for elements that are also included in FIG. 7B. In FIG. 10, the input data to the control device 20 comprises first measurement data 75A (cf. step 804), second measurement data 75B (cf. step 805), an initial dataset 872A, and property data 72B. As indicated, the second measurement data 75B comprises CRP values [Kp*] that have been measured at the above-mentioned measurement times. The initial dataset 872A comprises candidate values of Vp and Cpi. The PTM 72′ may be configured in accordance with FIG. 7C and is thus operable to generate a dataset 877A comprising estimated values for the solute concentrations, [Cpi], in the fluid within the PC. The estimated values are generated for time points that at least approximately match the measurement times of [Kp*]. In the illustrated example, the control device 20 further comprises a conversion module 877, which is configured to convert the estimated solute concentrations [Cpi] at each time point into a corresponding estimated conductivity [Kp]. The conversion module 877 may be configured to aggregate the conductivity contribution of all charged solutes, given their concentrations, while possibly also accounting for the effect of uncharged solutes, such as glucose and urea (if present). The conversion module 877 may thus be configured in accordance with well-known and standard equations, for example as described US2012/0018379 and WO2016/188950, which are incorporated herein by reference. The output data 877B of the conversion module 877, comprising [Kp], is received by a subtraction module 878, which is configured to compute the difference between corresponding values in [Kp] and [Kp*], i.e. between estimated conductivity values and measured conductivity values at the measurement times. The result is a sequence of difference values, represented as error data 873A in FIG. 10. The PFA 73′ is configured to operate on the error data 873A to generate candidate data 873B comprising an updated candidate Vp value, represented as Vpt in FIG. 10. The updated candidate Vp value replaces the corresponding value in the initial data set 872A. The PTM 72′ is configured to then operate on the candidate data 873B, and possibly on the candidate values for [Cpi] in the initial dataset 872A, to generate a new dataset 877A, comprising updated [Cpi]. As understood from the foregoing, the calculations and flow of data may continue until the PFA 73′ finds that a convergence criterion is fulfilled, for example that the error data 873A is small enough.

In some embodiments, the test cycle performed in step 803 of FIG. 8A is an “initial restricted probing cycle”, abbreviated IRPC in the following. The IRPC comprises, in sequence, a drain phase, a fill phase and a dwell phase. The IRPC may be performed, by the control device 20, at the beginning of a PD session and is “restricted” in that only a fraction of the maximum fill volume is withdrawn from the PC. The maximum fill volume may be measured for the patient or a generic value. Based on two or more CRP values measured during the IRPC in step 805, RVp may be determined in accordance with step 806.

An example of an IRPC and its use will be described with reference to FIG. 9, which shows intraperitoneal volume (Vp) as a function of time. It is assumed that Vp before start of the IRPC is unknown. The IRPC starts by a drain phase, in which the PDA 1 is operated to drain a restricted amount of fluid from the PC, designated by ΔVp. In some embodiments, ΔVp is set to limit the risk of drain pain. For example, ΔVp may be set equal to or less than a nominal residual volume, which may be determined for the patient or for a group of patients, for example based on historic data. In some embodiments, ΔVp is set to be less than 10%-25% of the maximum fill volume. In some embodiments, ΔVp is in the range of 50-400 mL or 100-300 mL. The drain phase is followed by a fill phase, in which fresh treatment fluid in infused into the PC. To avoid the risk of overfilling, the amount of infused fluid may be set equal to or less than ΔVp. During the IRPC, a first data sample Kpi0* is given by one or more measurements in the drain phase. A second data sample Kpi1* is taken a predefined time period (Ati) after completion of the fill phase. The time period Δti may be in the range of 0-20 minutes or 0-10 minutes. The second data sample Kpi1* may be obtained by the control device 20 operating the PDA 1 to draw a small fluid sample from the PC. Thus, Kpi1* results in a small decrease in fluid volume within the PC, as indicated in FIG. 9. After a short calculation time, at time point T1, RVp has been calculated based on the extracted volume (ΔVp), the infused volume, Kpi0* and Kpi1* (cf. step 806). As indicated in FIG. 9, the control device 20 may then proceed to perform an initial drain phase (IDP), followed by one or more fluid exchange cycles, as indicated by a subsequent fill phase (PD).

In FIG. 9, it is assumed that Vp is sufficient at the start of the IRPC. Should this not be the case, and ΔVp cannot be extracted from the PC, the initial drain phase of the IRPC will fail. If so, the control device 20 may perform an initial fill phase, in which an initial fill volume of treatment fluid is infused into the PC. The initial fill volume may be at least equal to ΔVp. A predefined time period after completion of the initial fill phase, for example Δti (above), the IRPC as shown in FIG. 9 is re-initiated.

The test cycle, for example IRPC in FIG. 9, may or may not be considered part of the session. If considered part of the session, the calculation of the set values in the method 400 will account for the contribution to the treatment target by the fluid exchange during the test period. This may be done by analogy with step 409C as described above (FIG. 4D). For example, step 401 of the method 400 may calculate an intermediate value of the treatment parameter(s) by use of the transport model (configured in step 802) and based on the first measurement data (obtained in step 804). The intermediate value represents how much of the treatment target that is achieved during the test period. Then, an updated treatment target is calculated by subtracting the intermediate value from the treatment target, TTV, obtained in step 401. The method 400 is then performed for the updated treatment target.

Hereinabove, it has been assumed that the variable Cₗᵢ in function ƒ4 is given by the nominal concentrations of solutes in fresh treatment fluid of a certain composition (in accordance with control parameter FC, FIG. 2C). However, accuracy may be improved by operating on actual concentrations, if available, or an estimation of the actual concentrations in the fresh treatment fluid. For example, ready-made treatment fluid is produced with relatively large tolerance limits of the ingredients. For example, the concentration of sodium may have a tolerance limit of +2.5%. This implies that conductivity may vary from batch to batch of treatment fluid. Thus, the use of nominal solute concentrations introduce errors into the calculations that are performed in the methods 400, 600A, 600B and 800. By measuring a CRP value of the fresh treatment fluid, and knowing the corresponding nominal CRP value, the nominal concentrations may be adjusted to better represent the actual concentrations. If the CRP values represent conductivity, this adjustment may be performed in different ways, as readily understood by the skilled person. In one example, the water fraction in the fresh treatment fluid may be calculated to minimize the difference between the nominal and measured conductivity while ensuring that the sum of charges in the treatment fluid is zero, whereupon the nominal concentrations are scaled by the water fraction. A CRP value of the fresh treatment fluid may be obtained by the control device 20 directing the fresh treatment fluid through the CRP sensor 15.

It is also conceivable to calculate RCpi based on a measured CRP value for treatment fluid extracted from the PC, for example during a drain phase. By measuring the CRP value, and knowing the corresponding nominal CRP value, the nominal concentrations may be adjusted to better represent the actual concentrations, resulting in RCpi. If the CRP values represent conductivity, this adjustment may be performed by analogy with the above-described adjustment for determination of Cₗᵢ.

Reverting to the calculation of updated transport properties in step 408 (FIG. 4A), this calculation may be performed by a structure similar to the structure in FIG. 10. To calculate an updated transport property, for example LpS or PSi, the PFA 73′ in FIG. 10 is modified to generate the candidate dataset 873B to comprise a candidate value of the transport property, which is then used by the PTM 72′ to generate the dataset 877A. Further, compared to FIG. 10, the initial dataset 872A comprises reference values of Vp and Cpi as well as initial values for the property data. These initial values are used by sub-modules 172, 173 (FIG. 7C) when the operation of the PTM 72′ is first started. The initial values may, for example, be obtained from the generic patient data 76B or the patient specific data 76C (FIG. 7A). Further details about the calculation of updated transport properties is given in Applicant's patent application PCT/EP2021/074288, which is incorporated in its entirety herein by reference.

Hereinabove, it has been assumed that the setup procedure 400A in FIG. 4A is performed to determine set values for an entire PD session, with the aim of achieving a treatment target at the end of the session. This type of “session control” may be modified to include more than one session, which means that the treatment target is given by a final value to be attained after plural sessions. In an implementation, the setup procedure 400A is performed to calculate set values for each session among the plural sessions, and the set values are the applied to control the PDA 1 to perform the respective session. Optionally, the set values may be recalculated before each session, to take into account the performance of preceding session(s) among the plural sessions.

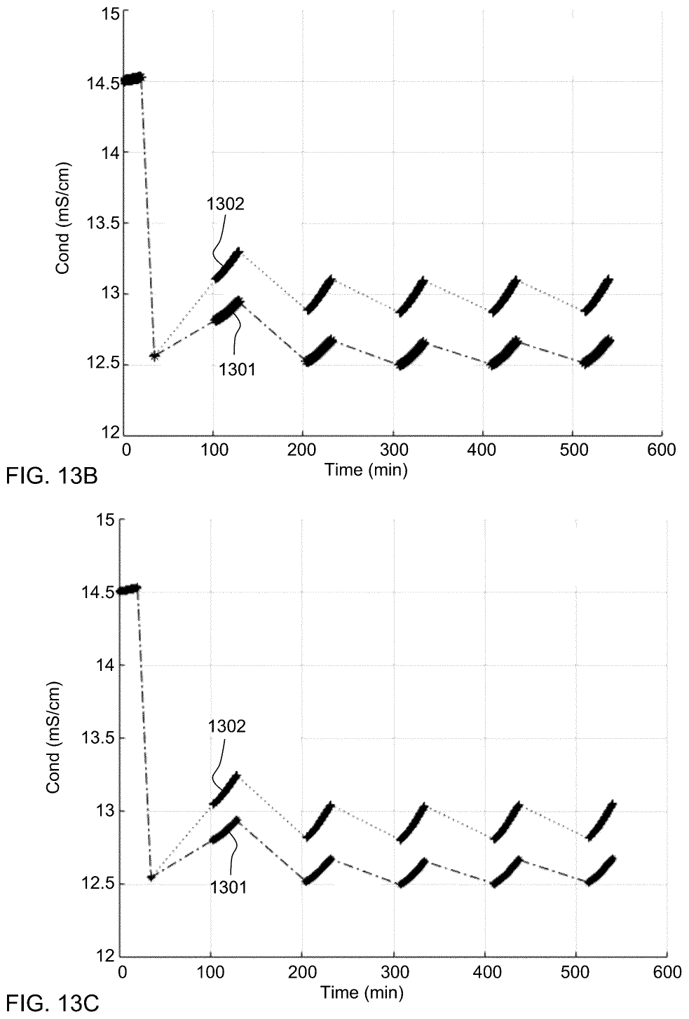

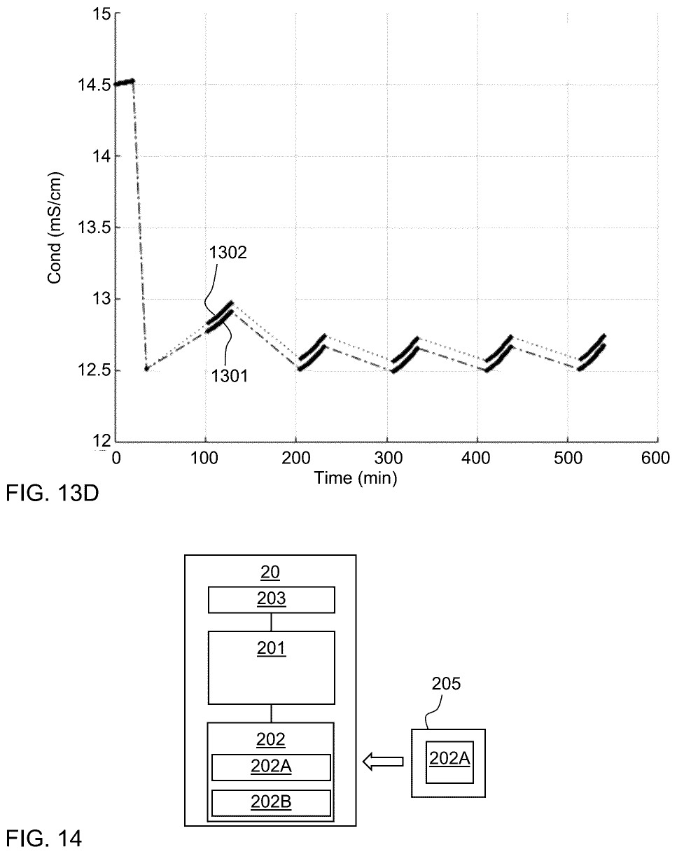

In a different variant, denoted “cycle control”, the setup procedure 400A is performed for an upcoming subset of a session (“session subset”), for example for individual cycles or groups of cycles. Here, the treatment target represents the desired change of the patient's physiological status during upcoming session subset, and the setup procedure 400A is repeated for consecutive session subsets throughout the session. The treatment target for the respective session subset may still be defined to achieve an overall treatment target for the complete session. One reason for implementing the cycle control is to ensure that Vp is kept within limits during the session. For example, as will be shown below with reference to FIGS. 13A-13D, errors in the values of the transport properties (step 402) may cause the actual Vp to deviate from the calculated Vp. If deterministic Vd extraction is performed, for example during tidal PD, a significant and unintentional change in Vres may occur during a PD session. By dividing the PD session into subsets, the deviation between actual Vp and calculated Vp will be reduced, and Vd may be dynamically determined to achieve a predefined Vres after each drain phase. The cycle control may be further improved by using the method 800 to calculate RVp for each session subset, which allows an actual Vres to be determined for individual cycles.

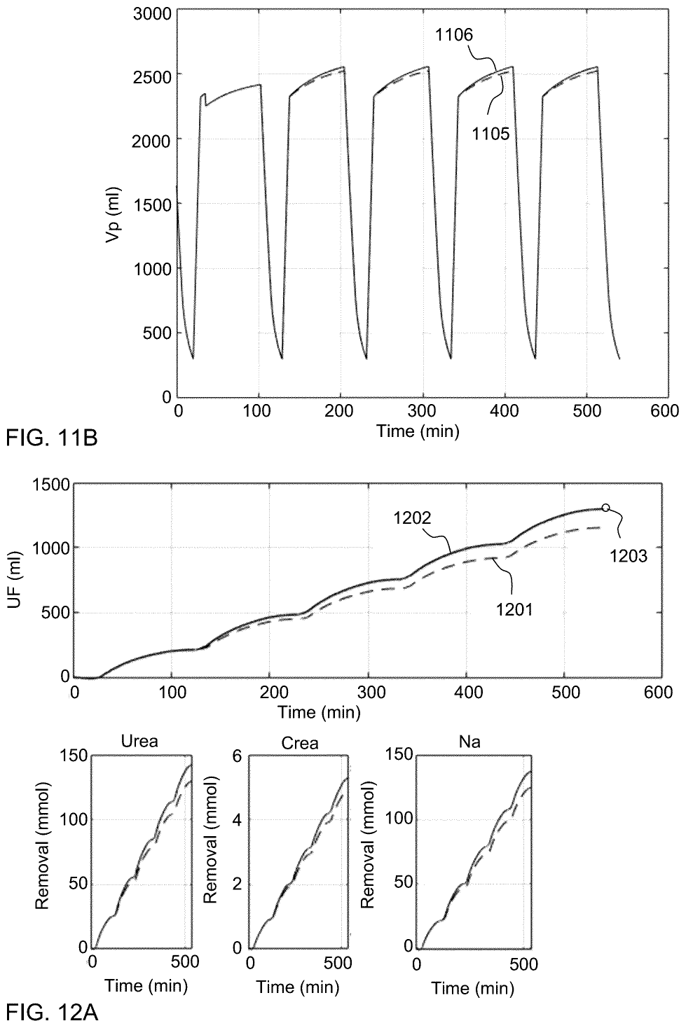

FIGS. 11-13 are presented to demonstrate the utility of the techniques described herein.

FIGS. 11A-11B represent a first scenario in which the concentration of glucose (cG) in the fresh treatment fluid is determined to achieve a target UF (UFV) of 1300 ml in a PD session, assuming that the fresh treatment fluid has the same cG throughout the session. All other control parameters are fixed. The reference values RVp and RCpi are calculated by treating the initial drain and the first cycle as a test cycle. The reference time point for the reference value is set at the end of the initial drain, i.e. RVp=Vres. The calculations assume that Vres is the same for all cycles. In the test cycle, the treatment fluid has a glucose concentration (cG) of 2.27%. The number of cycles, including the test cycle, is five. The fill volume in each cycle is 2000 ml. The calculations are started for a treatment fluid with cG=2.27%. The top graph in FIG. 11A shows UF as a function of time during the session, denoted “UF progression” in the following. The dashed line 1101 shows the UF progression resulting from a first execution of step 410 (FIG. 4B). As seen, the total UF at the end of the session deviates from the target UF (UFV=1300 ml). Steps 410-413 are therefore repeated. The solid line 1102 shows the UF progression resulting from the last execution of step 410, i.e. when the UF target 1103 is reached. The bottom graphs in FIG. 11A show corresponding progressions of solute removal for urea, creatinine and sodium. FIG. 11B is a graph of fluid volume (Vp) in the PC during the session. The dashed line 1105 represents fluid volume resulting from the first execution of step 410, and the solid line 1106 represents fluid volume resulting from the last execution of step 410. The sudden drop in fluid volume after 35 minutes is caused by a fluid sample (100 ml) being extracted from the PC for the purpose of measuring a CRP value (cf. Kpi1* in FIG. 9).

FIGS. 11A-11B demonstrate that it is possible to determine a glucose concentration in the treatment fluid that results in the target UF for a PD session. In the illustrated example, cG is 2.27% in the test cycle, and 2.46% in the following cycles.

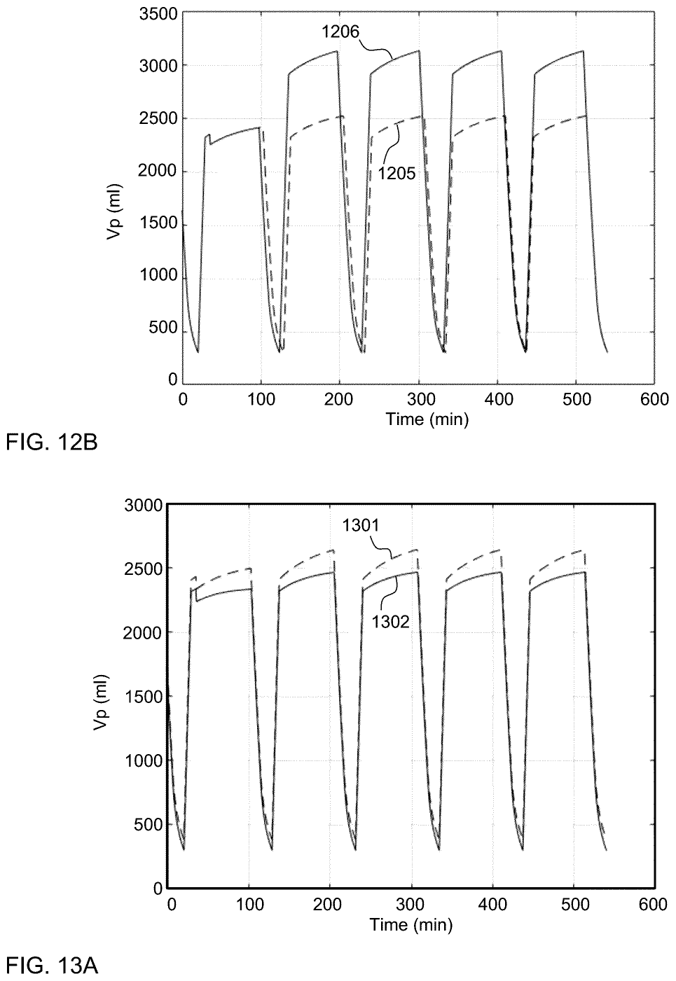

FIGS. 12A-12B represent a second scenario in which the fill volume (Vf) is determined to achieve a target UF (UFV) of 1300 ml in a PD session, assuming that the same fill volume is used in all cycles after the test cycle, which is defined and used as in the first scenario. All other control parameters are fixed. The fresh treatment fluid has cG=2.27%. The calculations are started for a fill volume of 2000 ml. FIG. 12A corresponds to FIG. 11A. The dashed line 1201 shows the UF progression resulting from the first execution of step 410. As seen, the total UF at the end of the session deviates from the target UF (UFV=1300 ml). The solid line 1202 shows the UF progression resulting from the last execution of step 410, i.e. when the UF target 1203 is reached. FIG. 12B corresponds to FIG. 11B. The dashed line 1205 represents fluid volume (Vp) resulting from the first execution of step 410, and the solid line 1206 represents fluid volume resulting from the last execution of step 410.

FIGS. 12A-12B demonstrate that it is possible to determine a fill volume that results in the target UF for a PD session. In the illustrated example, the fill volume is 2000 ml in the test cycle, and 2580 ml in the following cycles. FIGS. 13A-13D are provided to illustrate the sensitivity of calculations by the transport model to changes in the transport properties of the peritoneum. The underlying calculations in FIGS. 13A-13D are the same as for FIGS. 11A-11B, which means that the concentration of glucose (cG) in the fresh treatment fluid is determined to achieve a target UF (UFV) of 1300 ml. FIG. 13A shows fluid volume (Vp) in the PC as a function of time, and FIG. 13B shows CRP values (conductivity) in the fluid in the PC as a function of time during a session. The dark bands in FIG. 13B indicate conductivity that would be measured during the drain phases. Lines 1301 represent a first calculation based on default values of PSi and LpS, and lines 1302 represent a second calculation for 50% increase in PSi and 25% decrease in LpS. As seen, such a change in transport properties would result in a decrease in Vp and an increase in measured conductivity. It may be noted that the initial drain phase is used as a test cycle, causing the calculated conductivity to be inherently matched to the measured conductivity in this drain phase. The second calculation results in a total UF of 802 ml for the session. FIG. 13C corresponds to FIG. 13B and differs in that the second calculation is made for 50% increase in PSi and default LpS. As seen, the increase in measured CRP values is approximately the same as in FIG. 13B.

Although not shown, the second calculation also reveals that Vp is decreased, but the decrease is less than in FIG. 13A. The second calculation results in a total UF of 1067 ml for the session. FIG. 13D corresponds to FIG. 13B and differs in that the second calculation is made for default PSi and 25% decrease in LpS. As seen, the increase in measured CRP values is much smaller than in FIG. 13B. Although not shown, the second calculation also reveals that Vp is decreased, but the decrease is less than in FIG. 13A. The second calculation results in a total UF of 986 ml for the session.

The calculations represented by FIGS. 13A-13D reveal that a PD session performed in accordance with calculated set values may actually fail to meet its UFV, if a major change in LpS and/or PSi between sessions goes unnoticed. Such a major change may occur as a result of an infection in the patient, for example peritonitis. FIGS. 13B-13D indicate that one or more measured CRP values, for example measured during one or more drain phases, may be used for detecting a need to recalculate the transport properties, for example as described in relation to step 408 of method 400 (FIG. 4A). Further, FIG. 13A indicates that deviations in fluid volume (Vp) may also be used for detecting a need to recalculate the transport properties. For example, if the control device 20 implements the maximized Vd extraction, a need for recalculation may be triggered when Vres is found to increase between cycles. In accordance with FIG. 13A, the actual Vp in the PC will be lower than expected, which means that the actual Vd extracted during drain phases will be lower than expected. This means that Vres will increase for each cycle that is performed during the session. A temporary increase in Vres is not uncommon and may have other causes, for example a temporary dislocation of the access device 7′. Therefore, a recalculation of transport properties may be trigged if Vres increases (or equivalently, Vd decreases) for two or more consecutive cycles. In the context of the method 600A in FIG. 6A, such a change in Vres (or Vd) may be represented by DEV1 and trigger, in step 603, the update by step 408, in addition or instead of one or more deviating CRP values.

The structures and methods disclosed herein are applicable to any modality of automated peritoneal dialysis (APD), including but not limited to Continuous Cyclic Peritoneal Dialysis (CCPD), Intermittent Peritoneal Dialysis (IPD), Tidal Peritoneal Dialysis (TPD), Continuous Flow Peritoneal Dialysis (CFPD). All of these modalities involve at least one fluid exchange cycle that comprises a fill phase, a dwell phase and a drain phase.

The structures and methods disclosed herein may be implemented by hardware or a combination of software and hardware. In some embodiments, the hardware comprises one or more software-controlled computer resources. FIG. 14 schematically depicts a control device 20, which comprises one or more processors 201, computer memory 202, and a communication interface or circuit 203 for input and/or output of data. The processor(s) 201 and the computer memory 202 may be part of the control circuitry 20A (FIGS. 1 and 2C). The communication interface 203 may be configured for wired and/or wireless communication. As understood from the forgoing, the control device 20 may or may not be integrated in a dialysis machine. The processor(s) 201 may, for example, include one or more of a CPU (“Central Processing Unit”), a DSP (“Digital Signal Processor”), a GPU (“Graphics Processing Unit”), a microprocessor, a microcontroller, an ASIC (“Application-Specific Integrated Circuit”), a combination of discrete analog and/or digital components, or some other programmable logical device, such as an FPGA (“Field Programmable Gate Array”). A control program 202A comprising computer instructions is stored in the memory 202 and executed by the processor(s) 201 to implement logic that performs any of the methods, procedures, functions or steps described in the foregoing. The control program 202A may be supplied to the control device 20 on a computer-readable medium 205, which may be a tangible (non-transitory) product (e.g. magnetic medium, optical disk, read-only memory, flash memory, etc.) or a propagating signal. As indicated in FIG. 14, the memory 202 may also store control data 202B for use by the processor(s) 201, for example all or part of the property data 72B, the treatment history data 76A, the generic patient data 76B, the patient specific data 76C or the solute property data 76D (FIG. 7A).

While the subject of the present disclosure has been described in connection with what is presently considered to be the most practical embodiments, it is to be understood that the subject of the present disclosure is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results.

In the following, clauses are recited to summarize some aspects and embodiments as disclosed in the foregoing.

C1. A control device for operating a dialysis machine to perform a peritoneal dialysis treatment in relation to a patient, said dialysis machine being operable, based on control signals from the control device, to perform one or more fluid exchange cycles in relation to a peritoneal cavity of the patient to cause a transport of fluid and solutes through a peritoneal membrane in the peritoneal cavity, said control device comprising control circuitry configured to: obtain a target value of a treatment parameter for the peritoneal dialysis treatment; obtain at least one transport property of the peritoneal membrane of the patient; configure a transport model by use of said at least one transport property, the transport model defining the transport of fluid and solutes through the peritoneal membrane as a function of a plurality of control parameters for the peritoneal dialysis treatment performed by the dialysis machine; evaluate the transport model to determine set values of the plurality of control parameters to achieve the target value; and generate control signals for the dialysis machine in correspondence with the set values, said control signals causing the dialysis machine to perform the peritoneal dialysis treatment, wherein the control circuitry is further configured to: obtain first data comprising a measured value of a concentration-related parameter of fluid in the peritoneal cavity at one or more time points during the peritoneal dialysis treatment; obtain second data indicative of fluid flow of treatment fluid into and/or out of the peritoneal cavity during the peritoneal dialysis treatment; and calculate, by use of the transport model and based on the first data and the second data, at least one updated transport property of the peritoneal membrane of the patient.

C2. The control device of C1, wherein the control circuitry is further configured to reconfigure the transport model by said at least one updated transport property.

C3. The control device of C2, wherein the control circuitry is further configured to: evaluate the thus-reconfigured transport model to determine updated set values of the plurality of control parameters to achieve the target value, and generate updated control signals for the dialysis machine in correspondence with the updated set values.

C4. The control device of C2 or C3, wherein the control circuitry is further configured to present said at least one updated transport property on a user interface of the dialysis machine and/or store said at least one updated transport property in a memory associated with the control device.

C5. The control device of any one of C2-C4, wherein the control circuitry is further configured to: calculate a final value of the treatment parameter by use of the thus-reconfigured transport model and based on the second data; and present the final value on a user interface of the dialysis machine and/or store the final value in a memory associated with the control device.

C6. The control device of C1, wherein the control circuitry is further configured to: calculate, by use of the transport model and based on the first data and/or the second data, an expected value of an evaluation parameter for the fluid in the peritoneal cavity; determine a first deviation between the expected value and a corresponding value given by the first data or the second data; and selectively, based on the first deviation, calculate said at least one updated transport property of the peritoneal membrane of the patient.

C7. The control device of C6, wherein the control circuitry is configured to calculate, based on the second data, the expected value to represent the concentration-related parameter of fluid in the peritoneal cavity at said one or more time points during the peritoneal dialysis treatment, and determine the first deviation between the expected value and the measured value in the first data.

C8. The control device of C6 or C7, wherein the control circuitry is configured to: refrain from calculating said at least one updated transport property when the first deviation is between a first positive limit and a first negative limit; calculate said at least one updated transport property when the first deviation is larger than the first positive limit or smaller than the first negative limit; and generate an alert for an operator of the dialysis machine when the first deviation is larger than a second positive limit, which is larger than the first positive limit, or smaller than a second negative limit, which is smaller than the first negative limit.

C9. The control device of C8, wherein the control circuitry is further configured to refrain from calculating said at least one updated transport property when the first deviation causes the alert to be generated.

C10. The control device of C8 or C9, wherein the control circuitry is configured to, over time during the peritoneal dialysis treatment, perform at least one of: decreasing a first difference between the first positive limit and the first negative limit, or decreasing a second difference between the second positive limit and the second negative limit.

C11. The control device of any one of C6-C10, wherein the control circuitry is configured to: reconfigure the transport model by said at least one updated transport property and calculate, by use of the thus-reconfigured transport model, a final value of the treatment parameter; determine a second deviation between the final value and the target value; and selectively, based on the second deviation, evaluate the thus-reconfigured transport model to determine updated set values of the plurality of control parameters to achieve the target value, and generate updated control signals for the dialysis machine in correspondence with the updated set values.

C12. The control device of any preceding clause, wherein the control circuitry, when evaluating the transport model, is further configured to, repeatedly: calculate, by use of the transport model, time-resolved values of fluid volume and solute concentration in the peritoneal cavity for candidate set values of the plurality of control parameters; calculate a resulting value of the treatment parameter based on the time-resolved values of fluid volume and solute concentration; and modify one or more of the candidate set values until the resulting value fulfils a termination criterion or a time limit is reached.

C13. The control device of C12, wherein the control circuitry is configured to designate a first subset of the candidate set values as modifiable and a second subset of the candidate set values as fixed, and wherein said one or more of the candidate values is included in the first subset.

C14. The control device of C12 or C13, wherein the control circuitry is configured to: obtain a respective allowable range of said one or more of the candidate set values; and modify said one or more of the candidate set values within the respective allowable range.

C15. The control device of any preceding clause, wherein the plurality of control parameters comprises: a composition of a treatment fluid supplied to the peritoneal cavity during the peritoneal dialysis treatment, a number of fluid exchange cycles to be performed during the peritoneal dialysis treatment, an amount of treatment fluid supplied to the peritoneal cavity in a respective fluid exchange cycle during the peritoneal dialysis treatment, and a duration of the peritoneal dialysis treatment.

C16. The control device of any preceding clause, wherein the treatment parameter represents an accumulated amount of fluid removed from the patient during the peritoneal dialysis treatment, or an accumulated amount of one or more solutes removed from the patient during the peritoneal dialysis treatment.

C17. The control device of C16, wherein the control circuitry, to obtain the target value, is configured to obtain an initial weight of the patient before the peritoneal dialysis treatment, and a target weight of the patient at end of the peritoneal dialysis treatment, and calculate the target value for the accumulated amount of fluid based on the current weight and the target weight.

C18. The control device of C16, wherein the one or more solutes comprises urea or creatinine.

C19. The control device of any preceding clause, wherein the control circuitry, when evaluating the transport model, is further configured to obtain reference data comprising fluid volume and solute concentration in the peritoneal cavity at a reference time point, and calculate the set values by use of transport model and further based on the reference data.

C20. The control device of C19, wherein the control circuitry, to obtain the fluid volume in the peritoneal cavity at the reference time point, is configured to: operate the dialysis machine to perform a sequence of a drain phase and a fill phase during a measurement time period; obtain first measurement data indicative of fluid flow into and/or out of the peritoneal cavity, via a peritoneal access on the patient, during the measurement time period; obtain second measurement data comprising measured data samples representing solute concentration in the fluid volume in the peritoneal cavity at two or more time points during the measurement time period; calculate, by use of the transport model and based on the first measurement data, estimated data samples representing the solute concentration in the fluid volume at said two or more time points; and determine the fluid volume in the peritoneal cavity at the reference time point as a function of the measured data samples and the estimated data samples.

C21. The control device of C20, wherein the control circuitry is configured to, repeatedly until a convergence criterion is fulfilled or a time limit is reached: calculate time-resolved values of fluid volume and solute concentration in the peritoneal cavity by use of the transport model and based on the first measurement data and candidate values of the fluid volume and the solute concentration in the peritoneal cavity at a selected time point; determine the estimated data samples at the two or more time points based on the time-resolved values of solute concentration; and modify, based on a difference between the estimated data samples and the measured data samples, a candidate value of the fluid volume in the peritoneal cavity at the selected time point, wherein the control circuitry is configured to determine the fluid volume in the peritoneal cavity at the reference time point based on the calculated time-resolved values of fluid volume when the convergence criterion is fulfilled or the time limit is reached.

C22. The control device of C20 or C21, wherein the reference time point corresponds to a completion of the drain phase.

C23. The control device of any one of C20-C22, wherein the measured data samples comprise a first data sample taken during the drain phase and a second data sample taken subsequent to the fill phase.

C24. The control device of C23, wherein the first and second data samples are taken during an initial probing cycle which comprises the drain phase and the fill phase and in which a restricted amount of fluid in the peritoneal cavity is extracted in the drain phase, the restricted amount corresponding to a fraction of a maximum fill volume of the peritoneal cavity.

C25. The control device of C24, wherein the restricted amount is less than 10%-25% of the maximum fill volume.

C26. The control device of C24 or C25, wherein the restricted amount is in the range of 50-400 mL or 100-300 mL.

C27. The control device of any one of C24-C26, wherein the measurement time period is a part of the peritoneal dialysis treatment.

C28. The control device of any preceding clause, wherein the at least one transport property comprises a diffusion capacity of one or more solutes through the peritoneal membrane, and a filtration capacity of water through the peritoneal membrane.

C29. The control device of any preceding clause, wherein the transport model is a three-pore model for transport through the peritoneal membrane.

C30. The control device of any preceding clause, wherein the transport model is configured to account for ion transport by electrostatic force across the peritoneal membrane caused by differences in amounts of dissolved ions on opposite sides of the peritoneal membrane and reflection of large charged solutes by the peritoneal membrane.

C31. The control device of any preceding clause, wherein the control circuitry comprises a differential equation solver sub-module configured to calculate, from an initial time point to an end time point, fluid volume in the peritoneal cavity from the initial time point to the end time point including intermediate time steps, and to calculate solute concentration in the fluid volume from the initial time point to the end time point including the intermediate time steps.

C32. The control device of C31, wherein the differential equation solver sub-module is configured to calculate, for a respective time step, the fluid volume in the peritoneal cavity based on a preceding temporal change in the fluid volume, and to calculate, for the respective time step, a solute concentration in the fluid volume based on a preceding temporal change in the solute concentration.

C33. The control device of C32, wherein the control circuitry further comprises a first change computation system, which is configured to compute, for the respective time step, a temporal change in the fluid volume as a function of the solute concentration calculated by the differential equation solver sub-module for the respective time step, and as a function of the fluid volume in the peritoneal cavity calculated by the differential equation solver sub-module for the respective time step.

C34. The control device of C33, wherein the first change computation system comprises a first flow rate computation sub-module, which is configured to compute, for the respective time step, a flow rate of water through the peritoneal membrane as a function of the fluid volume calculated by the differential equation solver sub-module for the respective time step, and wherein the first change computation system further comprises a first change computation sub-module, which is configured to compute the temporal change in the fluid volume as a function of the flow rate of water through the peritoneal membrane for the respective time step.

35. The control device of C33 or C34, wherein the control circuitry further comprises a second change computation system, which is configured to compute, for the respective time step, a temporal change in the solute concentration as a function of the solute concentration calculated by the differential equation solver sub-module for the respective time step, and the fluid volume calculated by the differential equation solver sub-module for the respective time step.

36. The control device of C35, wherein the second change computation system comprises a second flow rate computation sub-module, which is configured to compute, for the respective time step, a flow rate of one or more solutes through the peritoneal membrane as a function of the solute concentration calculated by the differential equation solver sub-module for the respective time step, and the fluid volume calculated by the differential equation solver sub-module for the respective time step, and wherein the second change computation system further comprises a second change computation sub-module, which is configured to compute, for the respective time step, the temporal change in the solute concentration as a function of the flow rate of the one or more solutes through the peritoneal membrane for the respective time step, the flow rate of water through the peritoneal membrane for the respective time step, the solute concentration calculated by the differential equation solver sub-module for the respective time step, and the fluid volume calculated by the differential equation solver sub-module for the respective time step.

C37. An arrangement for performing peritoneal dialysis, comprising:
a fluid circuit that is connectable to a peritoneal access of a patient for conveying treatment fluid to and from a peritoneal cavity;
a dialysis machine configured to operate the fluid circuit; and
a control device in accordance with any one of C1-C36.

C38. A computer-implemented method of generating control signals causing a dialysis machine to perform a peritoneal dialysis treatment in relation to a patient, said peritoneal dialysis treatment comprising one or more fluid exchange cycles in relation to a peritoneal cavity of the patient to cause a transport of fluid and solutes through a peritoneal membrane in the peritoneal cavity, said method comprising: obtaining a target value of a treatment parameter for the peritoneal dialysis treatment; obtaining at least one transport property of the peritoneal membrane of the patient; configuring a transport model by said at least one transport property, the transport model defining the transport of fluid and solutes through the peritoneal membrane as a function of a plurality of control parameters for the peritoneal dialysis treatment performed by the dialysis machine; evaluating the transport model to determine set values of the plurality of control parameters to achieve the target value; and generating the control signals for the dialysis machine in correspondence with the set values; said method further comprising: obtaining first data comprising a measured value of a concentration-related parameter of fluid in the peritoneal cavity at one or more time points during the peritoneal dialysis treatment; obtaining second data indicative of fluid flow of treatment fluid into and/or out of the peritoneal cavity during the peritoneal dialysis treatment; and calculating, by use of the transport model and based on the first data and the second data, at least one updated transport property of the peritoneal membrane of the patient.

C39. A computer-readable medium comprising computer instructions which, when executed by one or more processors in the control device of any one of C1-C36, cause the one or more processors to perform the method of C38.

### Appendix A

The change in intraperitoneal volume is given by:

$$\frac{{dV}_{p}}{dt} = {J}_{v} ( t ) + {J}_{f} ( t ) - {J}_{d} ( t ) - {J}_{s} ( t ) - L \qquad ( 1 )$$

with J_{f}(t) being the flow rate during the fill phase(s), J_{d}(t) being the flow rate during the drain phase(s), Jₛ(t) being the fluid loss during sample extraction, and L being the lymphatic absorption, which may be assumed to be constant, for example at 0.3 mL/min.

The total fluid flow over the peritoneal membrane is the sum of the flow through the aquaporins (m1), small pores (m2) and large pores (m3):

$${J}_{v} ( t ) = {J}_{v , m 1} ( t ) + {J}_{v , m 2} ( t ) + {J}_{v , m 3} ( t ) = {\sum }_{m = 1}^{3} ( {A}_{f} ( t ) \cdot {α}_{m} \cdot {L}_{p} S \cdot ( Δ P ( t ) - RT {\sum }_{i = 1}^{N} {φ}_{i} \cdot {σ}_{m , i} \cdot ( {C}_{bi} - {C}_{pi} ( t ) ) ) ) \qquad ( 2 )$$

with αₘ being the fraction of hydraulic conductance for the respective pore type, which may be assumed to be α₁=0.02, α₂=0.90, and α₃=0.08, and with R being the ideal gas constant, T being the body temperature, and N being the number of different solutes.

The area factor, A_{f}(t), may be introduced to account for the fact that the effective area for exchange will depend on the volume of fluid in the peritoneal cavity. The area factor may be given by:

$${A}_{f} ( t ) = \frac{16.18 \cdot ( 1 - {e}^{- 0.00077 \cdot {V}_{p} ( t )} )}{13.3187}$$

The hydrostatic pressure difference between the peritoneal cavity and the capillaries, ΔP(t), may be calculated as ΔP(t)=P_{cap}(t)−IPP(t), where IPP(t) is the intraperitoneal pressure, which may be assumed to be a function of the intraperitoneal volume by:

$$IPP ( t ) = 4.7 + \frac{{V}_{p} ( t )}{690}$$

The capillary pressure, P_{cap}(t), may be set to be dependent on the mean arterial pressure, MAP, and the venous pressure, which may be assumed to be equal to IPP(t):

$${P}_{cap} ( t ) = \frac{1}{9} \cdot MAP + \frac{8}{9} \cdot IPP ( t )$$

The osmotic coefficient for the different solutes, φᵢ, may be given by tabulated values, and the reflection coefficient for the different solutes, σ_{m,i}, may be given by:

$${σ}_{m , i} = 1 - \frac{{( 1 - {λ}_{m , i} )}^{2} \cdot ( 2 - {( 1 - {λ}_{m , i} )}^{2} ) \cdot ( 1 - \frac{{λ}_{m , i}}{3} )}{1 - \frac{{λ}_{m , i}}{3} + \frac{2}{3} {λ}_{m , i}^{2}}$$

where λ_{m,i} is the quotient between the hydrodynamic radius of solute i, and the pore radius of the respective pore type (m=1,2,3). The hydrodynamic radius of the different solutes is an effective measure of the size, which is dependent on molecular mass/geometry as well as charge. It may also be noted that for aquaporins, σ_{1,i}=1.

The concentration differentials for each solute are dependent on the flow of solute and the dilution from fluid flow over the membrane as well as dilution during the fill phase(s):

$$\frac{{dC}_{pi}}{dt} = \frac{{J}_{i} ( t )}{{V}_{p} ( t )} - {C}_{pi} ( t ) \cdot \frac{{J}_{v} ( t ) + {J}_{f} ( t )}{{V}_{p} ( t )} + \frac{{C}_{Ii} \cdot {J}_{f} ( t )}{{V}_{p} ( t )} \qquad ( 3 )$$

with Cₗᵢ being the concentration of solute i in the fresh treatment fluid. The flow of solute i is given by:

$${J}_{i} ( t ) = {\sum }_{m = 2}^{3} {A}_{f} ( t ) \cdot {PS}_{i , m} \cdot {Pe}_{i , m} ( t ) \cdot \frac{{C}_{bi} - {C}_{pi} ( t ) \cdot {e}^{- {Pe}_{i , m} ( t )}}{1 - {e}^{- {Pe}_{i , m} ( t )}} \qquad ( 4 )$$

with Pe_{i,m}(t) being the dimensionless Peclet number (convective through diffusive) for solute i through pore type m, calculated by:

$${Pe}_{i , m} ( t ) = \frac{{J}_{v , m} ( t ) \cdot ( 1 - {σ}_{i , m} )}{{A}_{f} ( t ) \cdot {PS}_{i , m}} - {z}_{i} \cdot Δ E ( t )$$

with σ_{i,m} being the reflection coefficient of solute i at pore type m, which may be given by tabulated values. The last term is the electrostatic effect from the charge of the solute, zᵢ, and the potential, ΔE(t), across the membrane 30. However, as there will be no current between the peritoneal cavity 31 and the blood side 32:

$${\sum }_{i} ( {J}_{i , m 2} ( t ) + {J}_{i , m 3} ( t ) ) \cdot {z}_{i} = 0$$

with J_{i,m2}(t) and J_{i,m3}(t) being the flow of solute i through small pores (m2) and large pores (m3), respectively. This means that at each time point there will be a potential ΔE(t) that satisfies zero current. The magnitude of the potential, and thus Pe_{i,m}(t), may be determined by use of any suitable root-finding algorithm, for example a bracketing method such as a bisection method, or an iterative method such as by Newton's method or a Newton-like method.

1-39. (canceled)

40. A control device for operating a dialysis machine to perform a peritoneal dialysis treatment in relation to a patient, the dialysis machine being operable, based on control signals from the control device, to perform one or more fluid exchange cycles in relation to a peritoneal cavity of the patient to cause a transport of fluid and solutes through a peritoneal membrane in the peritoneal cavity, the control device comprising control circuitry configured to:
obtain a target value of a treatment parameter for the peritoneal dialysis treatment;
obtain at least one transport property of the peritoneal membrane of the patient;
configure a transport model by use of the at least one transport property, the transport model defining the transport of fluid and solutes through the peritoneal membrane as a function of a plurality of control parameters for the peritoneal dialysis treatment performed by the dialysis machine;
evaluate the transport model to determine set values of the plurality of control parameters to achieve the target value; and
generate control signals for the dialysis machine in correspondence with the set values, the control signals causing the dialysis machine to perform the peritoneal dialysis treatment,
wherein the control circuitry is further configured to:
obtain first data comprising a measured value of a concentration-related parameter of fluid in the peritoneal cavity at one or more time points during the peritoneal dialysis treatment;
obtain second data indicative of fluid flow of treatment fluid into and/or out of the peritoneal cavity during the peritoneal dialysis treatment; and
calculate, by use of the transport model and based on the first data and the second data, at least one updated transport property of the peritoneal membrane of the patient.

41. The control device of claim 40, wherein the control circuitry is further configured to reconfigure the transport model by the at least one updated transport property to generate a reconfigured transport model.

42. The control device of claim 41, wherein the control circuitry is further configured to: evaluate the reconfigured transport model to determine updated set values of the plurality of control parameters to achieve the target value, and generate updated control signals for the dialysis machine in correspondence with the updated set values.

43. The control device of claim 41, wherein the control circuitry is further configured to present the at least one updated transport property on a user interface of the dialysis machine and/or store the at least one updated transport property in a memory associated with the control device.

44. The control device of claim 41, wherein the control circuitry is further configured to:
calculate a final value of the treatment parameter by use of the reconfigured transport model and based on the second data; and
present the final value on a user interface of the dialysis machine and/or store the final value in a memory associated with the control device.

45. The control device of claim 40, wherein the control circuitry is further configured to:
calculate, by use of the transport model and based on the first data and/or the second data, an expected value of an evaluation parameter for the fluid in the peritoneal cavity;
determine a first deviation between the expected value and a corresponding value given by the first data or the second data; and
selectively, based on the first deviation, calculate the at least one updated transport property of the peritoneal membrane of the patient.

46. The control device of claim 45, wherein the control circuitry is configured to calculate, based on the second data, the expected value to represent the concentration-related parameter of fluid in the peritoneal cavity at the one or more time points during the peritoneal dialysis treatment, and determine the first deviation between the expected value and the measured value in the first data.

47. The control device of claim 45, wherein the control circuitry is configured to:
refrain from calculating the at least one updated transport property when the first deviation is between a first positive limit and a first negative limit;
calculate the at least one updated transport property when the first deviation is larger than the first positive limit or smaller than the first negative limit; and
generate an alert for an operator of the dialysis machine when the first deviation is larger than a second positive limit, which is larger than the first positive limit, or smaller than a second negative limit, which is smaller than the first negative limit.

48. The control device of claim 47, wherein the control circuitry is configured to, over time during the peritoneal dialysis treatment, perform at least one of: decreasing a first difference between the first positive limit and the first negative limit, or decreasing a second difference between the second positive limit and the second negative limit.

49. The control device of claim 48, wherein the control circuitry is configured to:
reconfigure the transport model by the at least one updated transport property to generate a reconfigured transport model and calculate, by use of the reconfigured transport model, a final value of the treatment parameter;
determine a second deviation between the final value and the target value; and
selectively, based on the second deviation, evaluate the reconfigured transport model to determine updated set values of the plurality of control parameters to achieve the target value, and generate updated control signals for the dialysis machine in correspondence with the updated set values.

50. The control device of claim 40, wherein the control circuitry, when evaluating the transport model, is further configured to, repeatedly:
calculate, by use of the transport model, time-resolved values of fluid volume and solute concentration in the peritoneal cavity for candidate set values of the plurality of control parameters;
calculate a resulting value of the treatment parameter based on the time-resolved values of fluid volume and solute concentration; and
modify one or more of the candidate set values until the resulting value fulfils a termination criterion or a time limit is reached.

51. The control device of claim 50, wherein the control circuitry is configured to designate a first subset of the candidate set values as modifiable and a second subset of the candidate set values as fixed, and wherein the one or more of the candidate values is included in the first subset.

52. The control device of claim 50, wherein the control circuitry is configured to: obtain a respective allowable range of the one or more of the candidate set values; and modify the one or more of the candidate set values within the respective allowable range.

53. The control device of claim 40, wherein the plurality of control parameters comprises: a composition of a treatment fluid supplied to the peritoneal cavity during the peritoneal dialysis treatment, a number of fluid exchange cycles to be performed during the peritoneal dialysis treatment, an amount of treatment fluid supplied to the peritoneal cavity in a respective fluid exchange cycle during the peritoneal dialysis treatment, and a duration of the peritoneal dialysis treatment.

54. The control device of claim 40, wherein the treatment parameter represents an accumulated amount of fluid removed from the patient during the peritoneal dialysis treatment, or an accumulated amount of one or more solutes removed from the patient during the peritoneal dialysis treatment.

55. The control device of claim 54, wherein the control circuitry, to obtain the target value, is configured to obtain an initial weight of the patient before the peritoneal dialysis treatment, and a target weight of the patient at end of the peritoneal dialysis treatment, and calculate the target value for the accumulated amount of fluid based on a current weight and the target weight.

56. The control device of claim 40, wherein the control circuitry, when evaluating the transport model, is further configured to obtain reference data comprising fluid volume and solute concentration in the peritoneal cavity at a reference time point, and calculate the set values by use of transport model and further based on the reference data.

57. The control device of claim 56, wherein the control circuitry, to obtain the fluid volume in the peritoneal cavity at the reference time point, is configured to:
operate the dialysis machine to perform a sequence of a drain phase and a fill phase during a measurement time period;
obtain first measurement data indicative of fluid flow into and/or out of the peritoneal cavity, via a peritoneal access on the patient, during the measurement time period;
obtain second measurement data comprising measured data samples representing solute concentration in the fluid volume in the peritoneal cavity at two or more time points during the measurement time period;
calculate, by use of the transport model and based on the first measurement data, estimated data samples representing the solute concentration in the fluid volume at the two or more time points; and
determine the fluid volume in the peritoneal cavity at the reference time point as a function of the measured data samples and the estimated data samples.

58. A computer-implemented method of generating control signals causing a dialysis machine to perform a peritoneal dialysis treatment in relation to a patient, the peritoneal dialysis treatment comprising one or more fluid exchange cycles in relation to a peritoneal cavity of the patient to cause a transport of fluid and solutes through a peritoneal membrane in the peritoneal cavity, the method comprising:
obtaining a target value of a treatment parameter for the peritoneal dialysis treatment;
obtaining at least one transport property of the peritoneal membrane of the patient;
configuring a transport model by the at least one transport property, the transport model defining the transport of fluid and solutes through the peritoneal membrane as a function of a plurality of control parameters for the peritoneal dialysis treatment performed by the dialysis machine;
evaluating the transport model to determine set values of the plurality of control parameters to achieve the target value; and
generating the control signals for the dialysis machine in correspondence with the set values;
the method further comprising:
obtaining first data comprising a measured value of a concentration-related parameter of fluid in the peritoneal cavity at one or more time points during the peritoneal dialysis treatment;
obtaining second data indicative of fluid flow of treatment fluid into and/or out of the peritoneal cavity during the peritoneal dialysis treatment; and
calculating, by use of the transport model and based on the first data and the second data, at least one updated transport property of the peritoneal membrane of the patient.

59. A computer-readable medium comprising computer instructions which,
when executed by one or more processors in a control device, cause the one or more processors to perform the method of claim 58,
wherein the control device is for operating the dialysis machine to perform the peritoneal dialysis treatment in relation to the patient, the dialysis machine being operable, based on control signals from the control device, to perform the one or more fluid exchange cycles in relation to the peritoneal cavity of the patient to cause the transport of fluid and solutes through the peritoneal membrane in the peritoneal cavity.
